(19) United States
(12) Patent Application Publication
(10) Pub. No.: US 2016/0017410 A1
(43) Pub. Date: Jan. 21, 2016

(54) HIGHLY MULTIPLEX SINGLE AMINO ACID MUTAGENESIS FOR MASSIVELY PARALLEL FUNCTIONAL ANALYSIS

(71) Applicants: Jay Shendure, Seattle, WA (US); Stanley Fields, Seattle, WA (US); Jacob Kitzman, Seattle, WA (US); Lea Starita, Seattle, WA (US)

(72) Inventors: Jay Shendure, Seattle, WA (US); Stanley Fields, Seattle, WA (US); Jacob Kitzman, Seattle, WA (US); Lea Starita, Seattle, WA (US)

(21) Appl. No.: 14/803,002

(22) Filed: Jul. 17, 2015

Related U.S. Application Data
(60) Provisional application No. 62/025,936, filed on Jul. 17, 2014.

(51) Int. Cl. C12Q 1/68; C12N 15/10

(52) U.S. Cl. CPC C12Q 1/6811; C12N 15/102; C12Q 1/6806; C12Q 1/6853

(57) ABSTRACT

Disclosed is a method for multiplexed mutagenesis of a target nucleotide sequence. The method entails generating, in parallel, a set of mutagenic oligonucleotide primers designed to cover all or part of the target nucleotide sequence, and reacting the set of mutagenic oligonucleotide primers with the target sequence in the presence of a polymerase to generate a mutant nucleotide sequence library, wherein each member of the mutant nucleotide sequence library comprises a full-length copy of the target nucleotide sequence having a unique programmed mutation derived from one member of the set of mutagenic oligonucleotide primers. Also disclosed are methods for generating a mutant nucleotide sequence library and for generating a mutant protein library.

### PRIORITY CLAIM

This application claims priority to U.S. Provisional Patent No. 62/025,936, filed Jul. 17, 2014, the subject matter of which is hereby incorporated by reference as if fully set forth herein.

### BACKGROUND

Saturation mutagenesis screens such as alanine scans [1] enable protein structure-function studies through directed inquiry into the functional consequences of mutations in individual amino acid residues. However, scaling these approaches to cover entire proteins is laborious and expensive, typically requiring the individual synthesis of mutagenic oligonucleotide primers for each target codon and their use in separate reactions. An alternative is random mutagenesis, e.g. error-prone PCR or doped oligonucleotide synthesis, but these methods fail to generate most amino acid substitutions that require multi-base changes.

Site-directed mutagenesis is an indispensable tool in sequence-structure-function studies, protein-engineering, and directed evolution [3]. The most widely used mutagenesis approaches are derivatives of the Kunkel method [4] and use oligonucleotides synthesized with the desired mutation, which prime on a wild-type template to copy the remaining wild-type sequence of interest. The parental template is marked with deoxyuracil bases (dUTPs) or, in more recent commercial approaches, dam methylation, allowing for its selective degradation [5]. These approaches traditionally target only one site at a time, such that many separate reactions must be performed in order to systematically mutagenize a protein sequence at every position, for instance to perform alanine scanning [1]. Further scaling this strategy to generate multiple distinct mutations at each site is even more labor-intensive. For example, Kato and colleagues individually constructed 2,314 point mutants of the tumor suppressor gene TP53, and serially assayed each mutant for its ability to transactivate a fluorescent reporter gene in a yeast model [6].

Recently introduced deep mutational scanning (DMS) approaches [2] take an alternative approach, in which large mutant libraries are first built in a bulk, randomized fashion. Digital counting via massively parallel DNA sequencing is then used to quantify the enrichment or depletion of individual mutants following functional selection on the complex library of mutants. In order to build the initial library of mutants, these approaches typically use doped oligonucleotide synthesis [7-9], in which a full-length region to be mutagenized is synthesized on controlled-pore glass (CPG) columns, with each phosphoramidite spiked with a small percentage of the other three, such that point mutations are randomly introduced along the length of the synthesized strand. However, single nucleotide deletions are common in CPG oligonucleotide synthesis due to incomplete incorporation in each step, limiting the length of mutant oligonucleotides that can be constructed without an unacceptably high rate of frame-shifting deletions. Furthermore, the minimum level of doping from most commercial vendors is 1%, which may be higher than desired. Error-prone PCR represents an alternative approach, but it requires empirical tuning to reach a desired mutational load and can be prone to bias [10]. Furthermore, a key limitation shared by doped oligonucleotide synthesis and error-prone PCR, when applied to protein-coding sequence, is that only a minority of the codon mutational space can be accessed through single-base mutations (e.g., 31% for p53).

Several recent approaches provide a degree of multiplexing for programmed mutagenesis. An extension of the original Kunkel method, P Funkel [11] uses pooled primer extension on a single-stranded circular phagemid template prepared from an E. coli host permissive of dUTP incorporation during DNA replication. Another approach, EMPIRIC [12], was used to mutagenize nine codons in a single reaction by inserting a cassette excisable by type IIS restriction digestion followed by replacement of this cassette with a mutagenized nine-codon cassette. These improved methods have recently enabled systematic measurement of point mutant fitness landscapes for portions of the yeast ubiquitin protein [13], a hepatitis C virus replication factor [14], and a bacterial beta-lactamase [15]. Despite such successes, these and other saturation codon mutagenesis methods remain laborious and cost-prohibitive, as they require individual synthesis of mutagenic primers or are limited in their scope by targeting only a few residues at a time, requiring serial tiling over the target.

### SUMMARY

In one aspect, this application relates to a method for multiplexed mutagenesis of a target nucleotide sequence. The method entails the steps of: a) generating, in parallel, a set of mutagenic oligonucleotide primers designed to cover all or part of the target nucleotide sequence; and b) reacting the set of mutagenic oligonucleotide primers with the target sequence in the presence of a polymerase to generate a mutant nucleotide sequence library. In step a), each member of the set of mutagenic oligonucleotide primers: i) is at least substantially complementary to a portion of the target nucleotide sequence, wherein the portion of the target nucleotide sequence is different from each member of the set, ii) includes a 5′ flanking adaptor sequence, a 3′ flanking adaptor sequence, or both, and iii) includes a unique programmed mutation near its center. In step b), each member of the mutant nucleotide sequence library includes a full-length copy of the target nucleotide sequence having a unique programmed mutation derived from one member of the set of mutagenic oligonucleotide primers.

In some embodiments, generating the set of mutagenic oligonucleotide primers entails: synthesizing and releasing a set of mutagenic oligonucleotides from a microarray; and amplifying and retrieving the set of mutagenic oligonucleotides using the 5′ flanking adaptor sequence or the 3′ flanking adaptor sequence. In some embodiments, the set of mutagenic oligonucleotide primers are designed to tile the target nucleotide sequence. In some embodiments, the 5′ flanking adaptor sequence or the 3′ flanking adaptor sequence is used to retrieve one or more copies of the target nucleotide sequence in the presence of the polymerase. In some embodiments, the unique programmed mutation includes one or more base changes, insertions, or deletions relative to the target nucleotide sequence. In other embodiments, the unique programmed mutation is a codon swap. In some embodiments, the target nucleotide sequence is a coding sequence. In some embodiments, the target nucleotide sequence is more than 10 codons in length. In some embodiments, each member of the set of mutagenic oligonucleotide primers is about 10 to 200 nucleotides in length.

In another aspect, this application relates to a method for generating a mutant nucleotide sequence library. The method entails the steps of: a) generating, in parallel, a set of mutagenic oligonucleotide primers that are at least substantially complementary to a portion of the target nucleotide sequence; b) annealing and extending the set of mutagenic oligonucleotide primers to and along a wild type sense template corresponding to the target nucleotide sequence, creating a set of sense megaprimers; c) amplifying the set of sense megaprimers using a pair of primers; d) annealing and extending the amplified set of sense megaprimers to and along a wild type antisense template corresponding to the target nucleotide sequence, creating a mutant nucleotide sequence library; and e) amplifying the members of the mutant nucleotide sequence library. In step a), each member of the set of mutagenic oligonucleotide primers includes: a unique programmed mutation, a 5′ flanking adaptor sequence, and a 3′ flanking adaptor sequence. In step b), the wild type sense template is marked for selective degradation. In step c), one of the primers recognizes and binds to the 5′ flanking adaptor sequence. In step d), each member of the mutant nucleotide sequence library includes a full-length copy of the target nucleotide sequence having a unique programmed mutation derived from one member of the set of mutagenic oligonucleotide primers.

In some embodiments, generating the set of mutagenic oligonucleotide primers entails: synthesizing and releasing a library of mutagenic oligonucleotides from a microarray, and amplifying and retrieving a subset of the library of mutagenic oligonucleotides using the 3′ flanking adaptor sequence, wherein the subset is the set of mutagenic oligonucleotide primers. In some embodiments, the wild type sense template and the wild type antisense template are degraded using a selective degradation agent following steps b) and d), respectively. In some embodiments, the method further includes removing the 3′ flanking adaptor sequence from the set of mutagenic oligonucleotide primers after step a) and before step b). In some embodiments, the method further includes removing the 5′ flanking adaptor sequence from the amplified set of sense megaprimers after step c) and before step d). In some embodiments, the set of mutagenic oligonucleotide primers are designed to tile the target nucleotide sequence. In some embodiments, the unique programmed mutation includes one or more base changes, insertions, or deletions relative to the target nucleotide sequence. In some embodiments, the unique programmed mutation is placed near the center of each mutagenic oligonucleotide primer. In some embodiments, the target nucleotide sequence is a coding sequence such as a coding sequence that is more than 10 codons in length. In some embodiments, each member of the set of mutagenic oligonucleotide primers is about 10 to 200 nucleotides in length.

In another aspect, this application relates to a method for generating a mutant protein library. The method entails the steps of: generating a mutant nucleotide sequence library using the method disclosed above, cloning each member of the mutant nucleotide sequence library into an expression plasmid, and expressing a mutated protein from each member of the mutant nucleotide sequence library that is cloned into an expression plasmid to generate the mutant protein library. In some embodiments, the mutant protein library is used to screen for the function of a mutant protein.

### BRIEF DESCRIPTION OF THE DRAWINGS

FIGS. 1A and 1B are schematics showing an overview of PALS mutagenesis according to some embodiments. FIG. 1A shows primers that are synthesized in parallel on a DNA microarray tiling a target sequence of interest and bearing programmed mutations (“X”), for instance to make specific codon replacements, to replace positions within degenerate sequence, or to for tiling deletions. Programmed mutations are introduced onto a wild-type template through sequential strand extension and PCR reactions. The final step yields full-length copies incorporating a single programmed mutation per copy. FIG. 1B shows mutant libraries that are cloned, with each clone receiving a unique molecular tag sequence. The library is subjected to hierarchical shotgun sequencing, with paired end reads interrogating the target gene insert from one end and the molecular tag from the other, to yield a set of consensus haplotypes and associated tags.

FIG. 2 shows coverage and uniformity of mutagenesis according to some embodiments. The fraction of amino acid substitutions observed as singleton mutations in three or more clones is plotted against the depth of coverage (clones per residue), for random subsets of increasing size from each library. Mutations are stratified by minimum required number of base pair edits to yield the corresponding amino acid substitution (1, 2, or 3). Protein length and final coverage by clones differ for each target, and dotted lines indicate equivalent clone coverage level across each, with the percent of all edit distance 1, 2 and 3 mutations covered at that shared level (1,646 clones per residue).

FIGS. 3A and 3B show en masse functional selection of Gal4 DBD PALS library highlights residues and mutations critical for transcriptional activity according to some embodiments. FIG. 3A shows sequence-function maps of mutation effect sizes across Gal4 DBD residues 2-65 (rows) for all programmed amino acid substitutions (columns; STOP: premature stop codon, Δ: inframe codon deletion) following outgrowth either without selection (top: SC-uracil, after 24 h) or under stringent selection for Gal4 (bottom: SC-uracil-histidine+1.5 mM 3-AT after 64 h). Heatmaps are shaded by the log 2-effect size, ranging from improved growth versus wild-type (red), equivalent to wild-type (white), to slower growth than wildtype (blue). Yellow and gray indicate the wild-type residue or insufficient data (minimum three tag-defined read groups per codon substitution required in the input library). FIG. 3B shows that functionally constrained residues overlap substantially with evolutionary conservation among Zn2/Cys6 family members (plotted in bits), including at the six domain-defining cysteines (indicated by arrows).

FIG. 4 shows validation of effects for selected Gal4 mutant alleles according to some embodiments. Plates were spotted with 10-fold serial dilutions of starting from numbers of cells carrying Gal4 1-196, either wild-type or with one of eight specifically introduced missense alleles. Growth on nonselective media (left) was uniform, while specific growth effects on selective media (right) qualitatively agreed with effect sizes observed by large scale selection (for each variant, top bar indicates effect size from non-selective culture and subsequent bars indicate effect size from selective outgrowth, Table SN, shaded as in FIG. 3A). Qualitative activity as measured by Johnston and Dover [20] is indicated alongside (+++, wild-type activity; hypo, hypomorphic; N.D., not determined).

FIG. 5 shows functional scores mapped to the Gal4 DBD structure according to some embodiments. The crystal structure for Gal4 residues 8-100, PDB accession 3COQ [22] is shown, with each amino acid (through residue 65) shaded by median effect size (excluding mutations to proline and premature truncation). Several key residues including zinc-coordinating cysteines, are highlighted with median effect size indicated.

FIG. 6 is a detailed schematic of PALS workflow according to some embodiments. Mutagenesis is carried out in eight steps, beginning with preparation of mutagenic primers from a DNA microarray. Next, strand extension, strand selection, and PCR are carried out twice to copy the wild-type sequence upstream and then downstream of each mutagenesis primer. A ninth step includes cloning the mutant library into plasmids for expression.

FIG. 7 shows purity and coverage for PALS and random mutagenesis according to some embodiments. For PALS and simulated random mutagenesis at various mutation rates, the percentage of possible amino acid substitutions carried on singleton clones (i.e., without any other missense mutations or frame-shift deletions) is plotted versus the percentage of possible substitutions carried on any clone. Simulated randomized mutagenesis was performed with different per-base substitution rates (point color indicates rate) and assuming various per-base deletion rates (rows; for Ube4b mutations introduced by doped oligonucleotide synthesis, a per-base deletion error rate of 8.9×10⁻⁴ was observed). Single-base substitution and deletion counts were sampled for each sequence from Poisson distributions with the indicated rates multiplied by sequence lengths, and missense and frame-shifting mutations were tallied. For substitutions, each of the three alternative bases was sampled with equal probability. Red points indicate the observed performance of PALS libraries in this study. Columns indicate the threshold minimum number of clones containing each mutation. Simulated clones were generated equal to the number of sequenced clones for A. Gal4 DBD (n=704,973) and B. p53 (n=646,939).

FIG. 8 shows regional coverage for PALS and doped oligonucleotide random mutagenesis according to some embodiments. Count of single coding mutant clones carrying each possible codon replacement is plotted against codon position. Each point represents a single codon replacement, shaded by number of base-pair differences.

FIG. 9 shows pairwise correlation scatterplots of effect size according to some embodiments. Per-mutation effect scores (log 2-scaled) are plotted for each pair of selection stringencies and timepoints. Black line indicates y=x, and Spearman rank correlation measure is inset.

FIG. 10 shows amino acid substitutions observed in orthologs are significantly less deleterious to Gal4 function than most mutations according to some embodiments. Mutation effect size distributions are shown in each of six selection timepoints (NONSEL, nonselective; others are selective). Premature truncations were excluded and remaining mutations are divided into three categories: (1, in blue) all substitutions observed in aligned GAL4 ortholog genes, (2, in green) substitutions not observed in GAL4 orthologs, at sites that did vary within the alignment, and (3) orange: substitutions at residues that were fixed among aligned GAL4 orthologs. Orthologs were identified by NCBI tblastx query of the wgs and genbank chromosomes databases at a cutoff of E<10-20, from genera Saccharomyces (n=11), Zygosaccharomyces (n=1), and Kluyveromyces (n=1). * denotes P<2.0×10⁻³, ** P<10⁻²⁰ and *** P<10⁻⁵⁰, Mann-Whitney U. Under every selective condition but not under non-selective outgrowth, mutations at fixed residues (group 3) were significantly more deleterious (more negative log 2 effect size) than mutations in either other group, and at residues that did vary among Gal4 orthologs, mutations that were not observed in those orthologs (group 2) were significantly less deleterious than those that were (group 1).

FIG. 11 shows examples of PALS amplification substrates and products according to some embodiments. Images of 6% TBE polyacrylamide gels stained by SYBR Gold (Invitrogen). Microarray-derived mutagenesis primers are shown following A. amplification (four different subsets of the library) and B. adaptor clipping (* indicates desired, 84 bp product). ladder-like′ amplification products following the first round of mutagenesis primer extension and adaptor-mediated PCR are shown (one replicate PCR product in each lane) in C. for Gal4 and in D. for p53. “25 bpl” 25 bp ladder (Invitrogen) and “100 bpl” is 100 bp ladder (NEB).

FIG. 12 shows a subassembly strategy according to some embodiments. Plasmid maps for PALS libraries constructed for A. Gal4 DBD and B. p53. The desired recircularization products are shown, with PCR primer names and amplicons inset.

FIG. 13 shows a subassembly validation example according to some embodiments. Read pileup and resulting subassembly consensus for a representative p53 clone (tag ACCCTAAGAGAATACGAGCT (SEQ ID NO:19)), consensus haplotype K120L). Shown below are the capillary sequencing traces through the insert showing the K120L mutation (middle), and the clone-identifying barcode (bottom).

FIG. 14 (SEQ ID NOS:1-38) is a table showing Sanger-sequencing validation of subassembled clones according to some embodiments. A total of 40 clones were individually picked and Sanger sequenced across the targeted ORF and associated clone tag, using two reads (Gal4 DBD) or four reads each (p53). Two clones missing from the subassemblies had partially truncated tag sequences (both had single codon replacements with no additional mutations) and one was excluded after failing the allele fraction filter during subassembly. Each of the remaining 37 clone sequences was perfectly concordant with the subassembly consensus sequence bearing the same tag (i.e., no missing or extra mutations). ND, not determined; syn, synonymous mutation.

FIG. 15 is a table showing Gal4 selection cultures and timepoints according to some embodiments.

FIG. 16 is a table showing a comparison of previously reported activities for GAL4 mutant alleles with effect size measurements in this study in accordance with one embodiment. Effect sizes measured in the present study are given as rescaled log 2 values (wild-type=0). Jelicic et al (2013) measured transcriptional activity using a GAL-responsive MEL1 reporter and introduced mutations into a Gal4 fragment containing residues 1-100+840-881. Ferdous et al (2008) performed a similar assay using Gal4 1-147+799-1082. Johnston and Dover (1988) screened Gal-mutant alleles within the full-length, native Gal4 locus for activity using a LacZ reporter. ND, not determined.

FIG. 17 is a table showing a comparison of oligonucleotide synthesis cost, per targeted residue, between PALS and other programmed mutagenesis techniques according to some embodiments. Cost estimates based upon publicly available list prices for 12 k feature 90mer array (CustomArray, Inc.) and 60mer synthesis at the smallest available scale (Integrated DNA Technologies).

FIG. 18 is a table showing a summary of sequencing performed according to some embodiments. Summary of reads collected for PALS library sequencing and counting

FIG. 19 (SEQ ID NOS:39-96) is a table of primers used in the methods according to some embodiments.

FIG. 20 is a table indicating the PCR conditions used in accordance with some embodiments.

### DETAILED DESCRIPTION

Methods for multiplexed mutagenesis of a target nucleotide sequence and methods are provided herein. Such methods may be used to generate mutant nucleotide sequence libraries and mutant protein libraries for use in other applications such as sequence-structure-function studies, protein-engineering, directed evolution, or any other suitable application.

To overcome the limitations of previously used methods, a method which may be referred to herein as PALS (“programmed allelic series”) was developed, which combines low-cost, microarray-based DNA synthesis of alleles with single-tube overlap extension mutagenesis in order to introduce one and only one mutation per cDNA template in a massively parallel fashion.

According to the embodiments described herein, this application relates to a method for multiplexed mutagenesis of a target nucleotide sequence. The target nucleotide sequence may be any suitable DNA or RNA sequence, including any portion of a gene or RNA molecule (e.g., mRNA, tRNA, siRNA, shRNA, miRNA), and may include a coding sequence, a non-coding sequence, or a sequence that includes a portion of a coding sequence and a portion of a non-coding sequence.

The target nucleotide sequence may be of any length. In certain embodiments, the target nucleotide sequence is more than 10 codons in length. In other embodiments, the target nucleotide sequence is more than 20 codons in length, more than 30 codons in length, more than 40 codons in length, more than 50 codons in length, more than 60 codons in length, more than 70 codons in length, more than 80 codons in length, more than 90 codons in length, more than 100 codons in length, more than 150 codons in length, more than 200 codons in length, more than 250 codons in length, more than 300 codons in length, more than 350 codons in length, more than 400 codons in length, more than 450 codons in length, more than 500 codons in length, more than 1000 codons in length.

The method includes a step of generating, in parallel, a set of mutagenic oligonucleotide primers designed to cover all or part of the target nucleotide sequence. In certain aspects, the step of generating the set of mutagenic oligonucleotide primers is accomplished by synthesizing the entre set of primers in parallel, i.e., at the same time during a single reaction instead of one-by-one or in small groups. In some embodiments, the synthesis is by microarray. in

Each member of the set of mutagenic oligonucleotide primers is at least substantially complementary to a portion of the target nucleotide sequence. “Substantially complementary,” as used herein means that a first nucleic acid molecule is (1) entirely or traditionally complementary to a second nucleic acid molecule, i.e., when the first and second nucleic acid molecules hybridize to each other to form base pairs between traditional nucleotide bases: adenine is matched to thymine (DNA) or uracil (RNA), and guanine is matched to cytosine, (2) substantially or non-traditionally complementary to a second nucleic acid molecule, wherein one or more bases are paired with a base that is not a traditional pairing (e.g. IUPAC matched bases) or a non-traditional or synthetic base when the two molecules hybridize to one another.

Each mutagenic oligonucleotide primer is generally between about 10 to about 200 nucleotides (nt) in length, but may be any suitable length, including, but not limited to, about 10 to about 100 (nt) in length, about 50 to about 100 (nt) in length, about 60 to about 100 (nt) in length, about 70 to about 100 (nt) in length, about 80 to about 100 (nt) in length, about 90 to about 100 (nt) in length, about 50 to about 150 (nt) in length, about 100 to about 200 (nt) in length, about 100 to about 150 (nt) in length, about 150 to about 200 (nt) in length, about 10 (nt) in length, about 20 (nt) in length, about 30 (nt) in length, about 40 (nt) in length, about 50 (nt) in length, about 60 (nt) in length, about 70 (nt) in length, about 80 (nt) in length, about 90 (nt) in length, about 100 (nt) in length, about 110 (nt) in length, about 120 (nt) in length, about 130 (nt) in length, about 140 (nt) in length, about 150 (nt) in length, about 160 (nt) in length, about 170 (nt) in length, about 180 (nt) in length, about 190 (nt) in length, about 200 (nt) in length, or any other suitable length.

Although not a requirement, the primer is generally shorter than the target sequence. Thus, each mutagenic oligonucleotide primer should correspond to a different portion of the target nucleotide sequence. This allows the set of primers to cover the entire target nucleotide sequence. In certain aspects, the primers are designed to tile the target nucleotide sequence.

A mutagenic oligonucleotide primer generated in accordance with the methods described herein also includes a 5′ flanking adaptor sequence, a 3′ flanking adaptor sequence, or both a 5′ flanking adaptor sequence, a 3′ flanking adaptor sequence. These adaptor sequences are not present in the target nucleotide sequence, but are used to retrieve a set or subset of nucleotides generated using the methods described herein. For example, the adaptor sequences are used to retrieve one or more copies of the target nucleotide sequence in the presence of a polymerase (e.g., during a PCR reaction), or may be used to retrieve a set or subset of mutagenic oligonucleotides that have been synthesized in parallel.

Further, each mutagenic oligonucleotide primer generated in accordance with the methods described herein includes a unique programmed mutation such that each primer has a different mutation. In some aspects, the mutation is near the center of the primer. The mutation may include, but is not limited to, one or more base changes, insertions, or deletions relative to the target nucleotide sequence. In one aspect the unique programmed mutation is a codon swap.

The methods described herein also include a step of reacting the set of mutagenic oligonucleotide primers generated in the previous step with the target sequence in the presence of a polymerase to generate a mutant nucleotide sequence library.

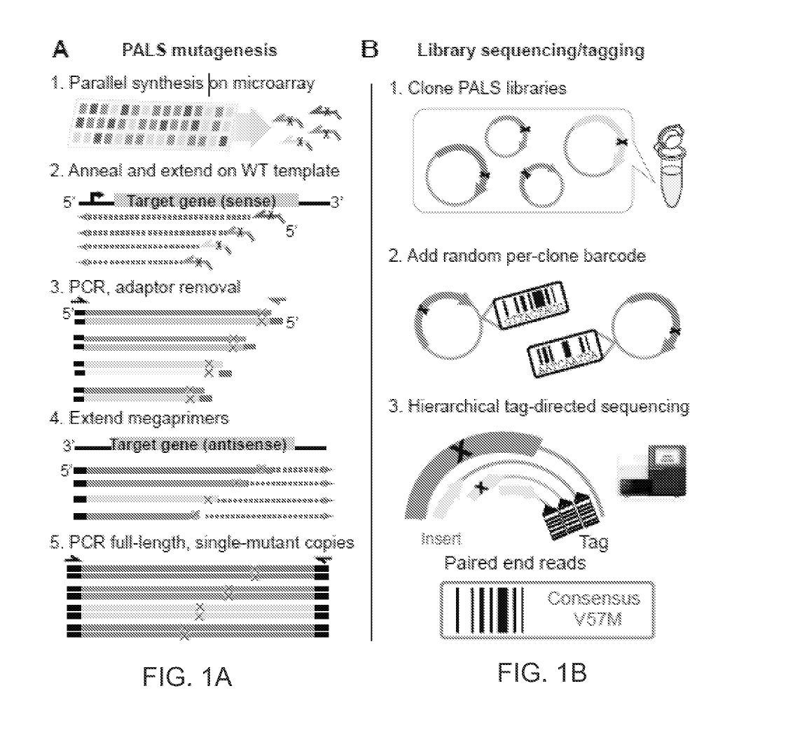

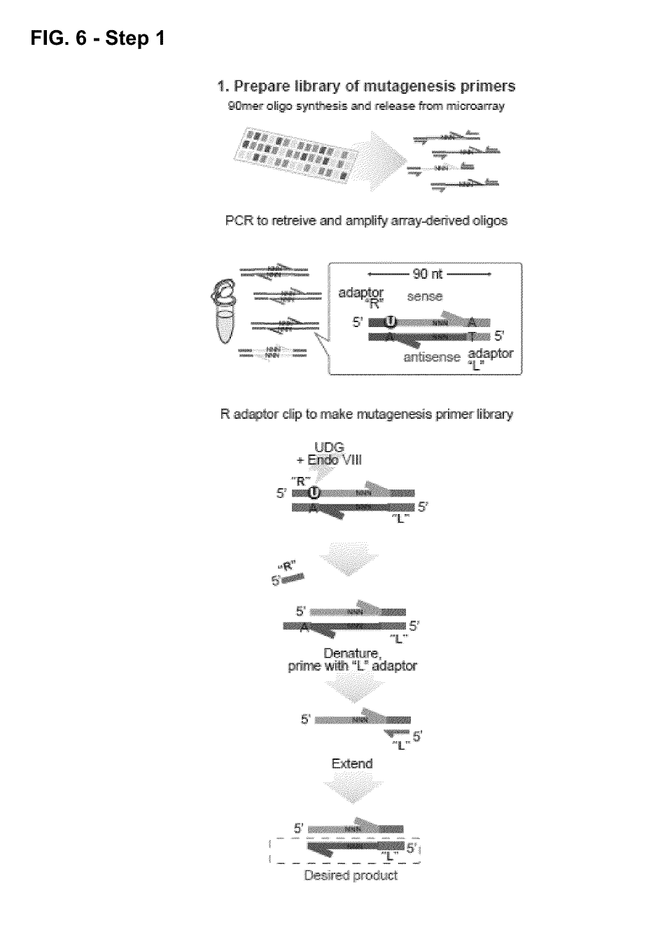

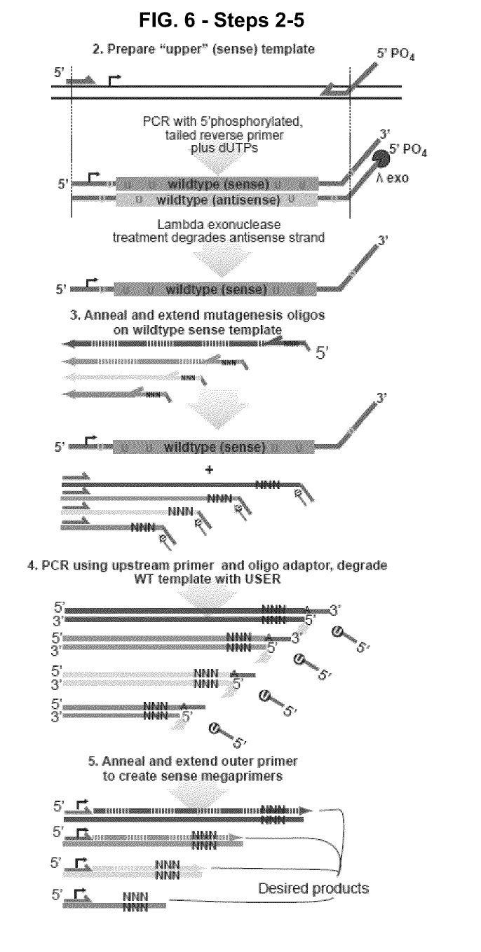

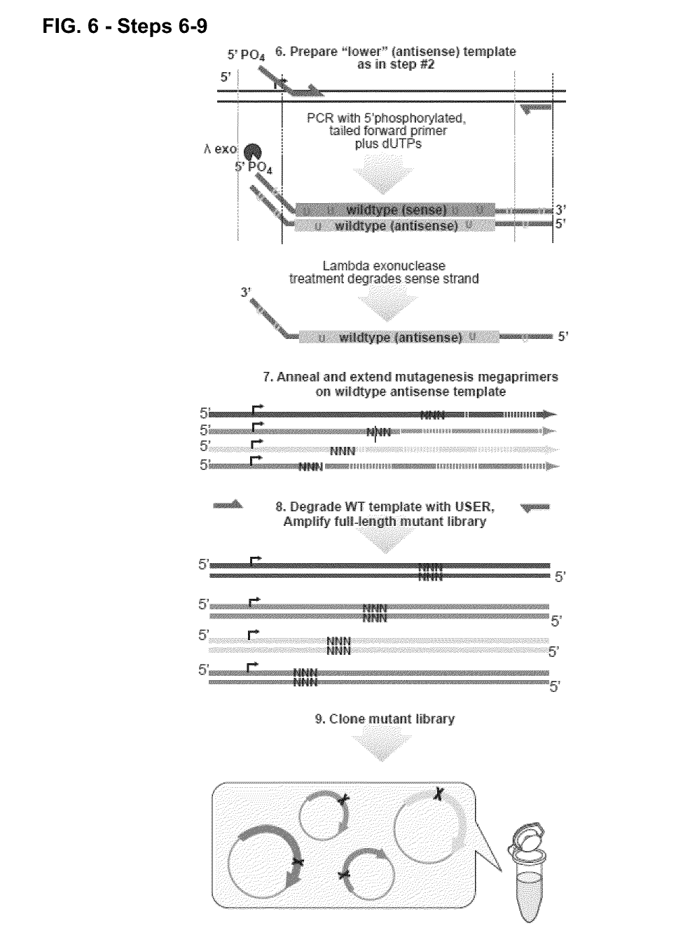

The reactions between the set of mutagenic oligonucleotide primers and the target sequence may include reactions (e.g., PCR, which is a reaction in the presence of a polymerase) with an antisense strand of the target nucleotide sequence, a sense strand of the target nucleotide sequence, or both. In certain embodiments, these reactions are described in FIGS. 1 and 6. The reactions result in the generation of a mutant nucleotide sequence library. According to the embodiments described herein, each member of the mutant nucleotide sequence library includes a full-length copy of the target nucleotide sequence that has a unique programmed mutation derived from one member of the set of mutagenic oligonucleotide primers.

In one embodiment, a method for generating a mutant nucleotide sequence library is provided. The method include the steps of: a) generating, in parallel, a set of mutagenic oligonucleotide primers that are at least substantially complementary to a portion of the target nucleotide sequence; b) annealing and extending the set of mutagenic oligonucleotide primers to and along a wild type sense template corresponding to the target nucleotide sequence, creating a set of sense megaprimers; c) amplifying the set of sense megaprimers using a pair of primers; d) annealing and extending the amplified set of sense megaprimers to and along a wild type antisense template corresponding to the target nucleotide sequence, creating a mutant nucleotide sequence library; and e) amplifying the members of the mutant nucleotide sequence library. In step a), each member of the set of mutagenic oligonucleotide primers includes: a unique programmed mutation, a 5′ flanking adaptor sequence, and a 3′ flanking adaptor sequence. In step b), the wild type sense template is marked for selective degradation. In step c), one of the primers recognizes and binds to the 5′ flanking adaptor sequence. In step d), each member of the mutant nucleotide sequence library includes a full-length copy of the target nucleotide sequence having a unique programmed mutation derived from one member of the set of mutagenic oligonucleotide primers. This method is illustrated in FIG. 6 according to one aspect of the embodiment.

In some embodiments, generating the set of mutagenic oligonucleotide primers includes: synthesizing and releasing a library of mutagenic oligonucleotides from a microarray, and amplifying and retrieving a subset of the library of mutagenic oligonucleotides using the 3′ flanking adaptor sequence, wherein the subset is the set of mutagenic oligonucleotide primers. In some embodiments, the wild type sense template and the wild type antisense template are degraded using a selective degradation agent following steps b) and d), respectively. In some embodiments, the method further includes removing the 3′ flanking adaptor sequence from the set of mutagenic oligonucleotide primers after step a) and before step b). In some embodiments, the method further includes removing the 5′ flanking adaptor sequence from the amplified set of sense megaprimers after step c) and before step d).

In another embodiment, the methods described herein are illustrated generally in FIG. 1, and begins with DNA microarray synthesis of mutagenic primers designed to tile a coding sequence of interest, with the mutation, e.g., a codon swap, placed near the center (FIG. 1A, step 1). These are released from the microarray to yield a complex mixture of oligonucleotides in solution. Each primer library is designed with flanking adaptor sequences, allowing specific subsets to be retrieved from the microarray-derived oligonucleotide library by PCR. After the downstream adaptors are removed (FIG. 6), the resulting pools of tailed primers are annealed and extended along a linear dUTP-containing template corresponding to the wild-type sense strand (step 2), which is then degraded by treatment with uracil-DNA-glycosylase (UDG) and exonuclease VIII. The “ladder-like” extension reaction product is PCR-amplified using a forward primer upstream of the gene, and a reverse primer corresponding to the adaptor sequence at the 5′ end of each mutagenic primer (step 3). Following this step, the remaining adaptor sequence is clipped, and the resulting mutagenized megaprimer is annealed and extended along the antisense strand of the wild-type template (step 4). Residual wildtype template copies are again degraded by UDG treatment, and the full-length mutant library of mutant cDNAs is enriched by PCR (step 5) and may be cloned into plasmids for expression.

To assess coverage of the programmed mutations and the off-target mutation rate, the PALS library resulting from this method is sequenced. Provided sufficient depth, shotgun sequencing of the complex library of mutant clones may sensitively detect all the introduced mutations. However, existing sequencing technologies still produce reads that are too short to cover full-length ORFs or even individual domains, such that one is unable to phase multiple mutations on the same clone when they are separated by more than the read insert size. Consequently, for instance, a neutral allele could be wrongly counted as highly deleterious when coupled to a loss-of-function allele elsewhere on the same clone. Some sequencing platforms (e.g., Pacific Biosciences) are capable of longer reads but these currently come at the expense of high per-base error rates (up to 15%), such that they are not readily suited to unambiguously identifying clones that contain only a programmed mutation.

For these reasons, tag-directed hierarchical sequencing or sub-assembly [16] were adopted as a way to validate the composition and quality of PALS libraries. In this approach (FIG. 1B), a library of mutants is tagged with a degenerate barcode such that each cloned cDNA molecule is coupled to a distinct random k-mer (k=16 or 20), hereafter referred to as the “tag”. Paired-end reads are then obtained from the tagged clones, wherein one end (fixed) reports the tag sequence, and the other end (shotgun) is derived randomly from the insert. The shotgun reads are then grouped by tag to yield a consensus haplotype that is longer than the constituent reads, and that also corrects random sequencing errors. In addition to enabling full-length sequencing of individual cDNA clones that are longer than the read-length of the sequencing platform, a further advantage of this approach is that to quantify allelic enrichment or depletion following function-dependent selection, it is only necessary to sequence and count tags rather than the entire cDNA.

The methods described herein may be performed with reagents and/or platforms that may be assembled in a kit, or available separately. For example, the reagents and materials described in the methods below may be formulated and assembled in a single kit to allow a user to perform the method by purchasing everything that is needed in a single place.

The mutant nucleotide sequence library generated in accordance with the methods described above may be used to generated a mutant protein library that can be used to assess the function of mutant proteins as discussed in the examples below. As such a method for generating a mutant protein library is provided in accordance with the methods described above. Such a method includes steps of: generating a mutant nucleotide sequence library using the method disclosed above, cloning each member of the mutant nucleotide sequence library into an expression plasmid, and expressing a mutated protein from each member of the mutant nucleotide sequence library that is cloned into an expression plasmid to generate the mutant protein library.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Sequence data reported herein have been deposited in the Sequence Read Archive (SRA), www.ncbi.nlm.nih.gov/sra (accession code SRA169378). Further, all references cited in the disclosure are hereby incorporated by reference in their entirety, as if fully set forth herein.

### Examples

Saturation mutagenesis screens such as alanine scans [1] enable protein structure-function studies through directed inquiry into the functional consequences of mutations in individual amino acid residues. However, scaling these approaches to cover entire proteins is laborious and expensive, typically requiring the individual synthesis of mutagenic oligonucleotide primers for each target codon and their use in separate reactions. An alternative is random mutagenesis, e.g. error-prone PCR or doped oligonucleotide synthesis, but these methods fail to generate most amino acid substitutions that require multi-base changes. To overcome these challenges, PALS (“programmed allelic series”), a highly multiplexed, site-directed mutagenesis approach that leverages massively parallel oligonucleotide synthesis on microarrays was developed. PALS is demonstrated by using single reactions to introduce every possible single-codon mutation into the DNA-binding domain (DBD) of the yeast transcription factor Gal4 (64 amino acid residues) and the human tumor suppressor p53 (393 residues). Full-length, haplotype-resolved sequencing of the resulting 1.35 million clones identified 99.9% and 93.5% of the programmed mutations as singleton mutations on an otherwise wild-type background in each respective gene. Subjecting the Gal4 PALS library to an in vivo selection for transcriptional activation demonstrated that nearly a third of the DBD is intolerant to mutation. Additionally, several mutations in the linker domain that increased function are identified in the assay, possibly by orienting the flanking domains more favorably for transcriptional activation. Fully covering codon mutation space with single amino acid changes facilitates a more finely resolved landscape of protein-coding functional constraint. This method may also be useful for massively multiplexed biochemical characterization of clinically observed missense variants of unknown significance in disease associated genes.

Methods

Mutagenic Primer Preparation.

Mutagenic primers were electrochemically synthesized on a 12,432-feature programmable DNA microarray and released into solution by CustomArrray, Inc [34]. For Gal4 (GI #6325008), codons 2-65 were each replaced with the optimal codon in S. cerevisae corresponding to one of the 19 other amino acids {Nakamura:2000 wk}, a stop codon (TAA), or an in-frame deletion, for a total of 1,344 oligos each spotted in duplicate. For p53 (GI #120407068), codons 1-393 were replaced with fully degenerate bases (“NNN”), such that each primer molecule synthesized within a single spot on the array includes a different one of 64 randomized codons, with each of the 393 oligos spotted in triplicate.

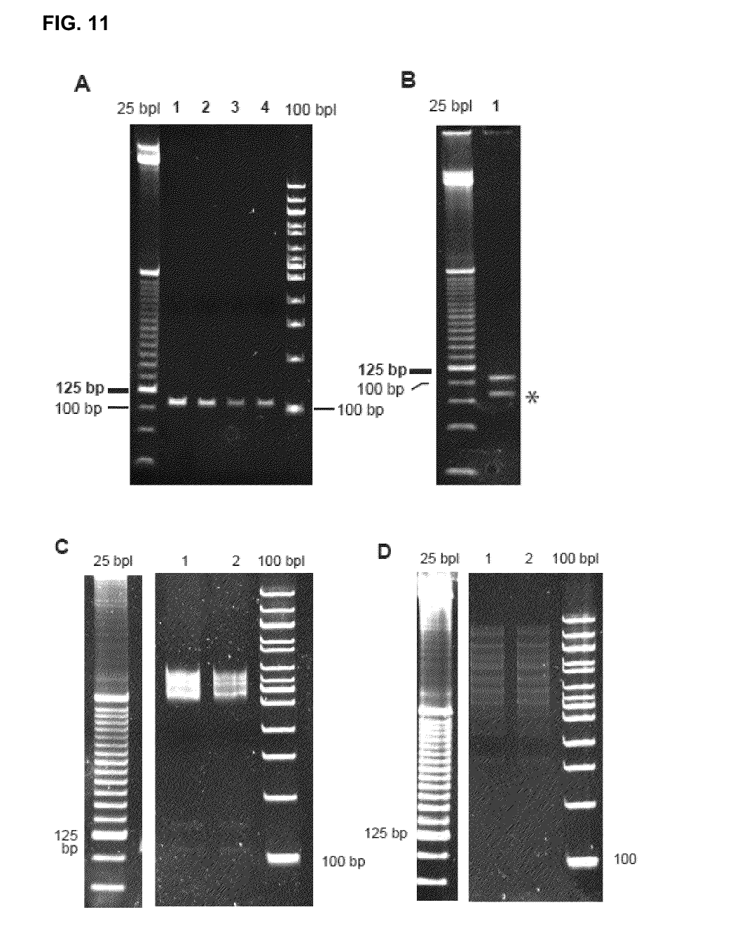

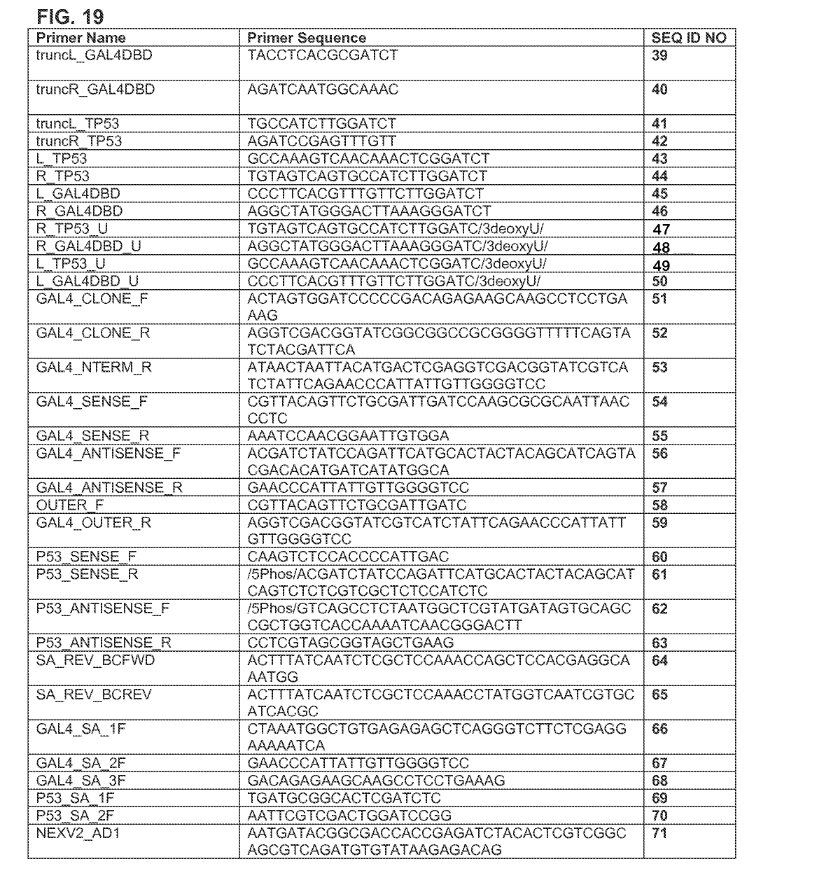

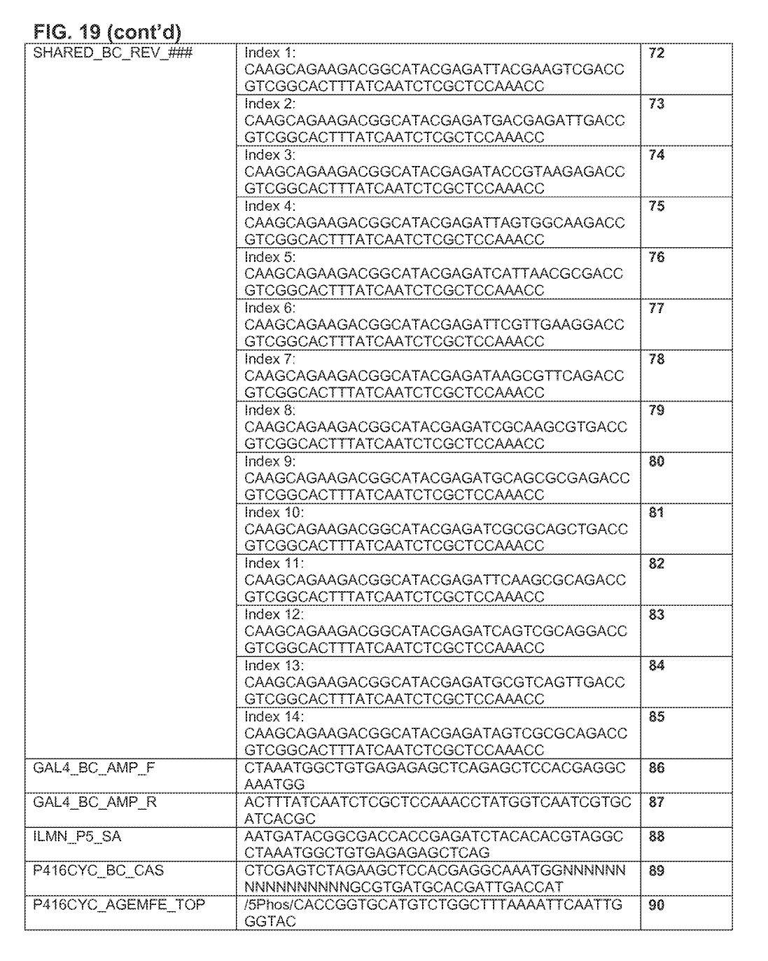

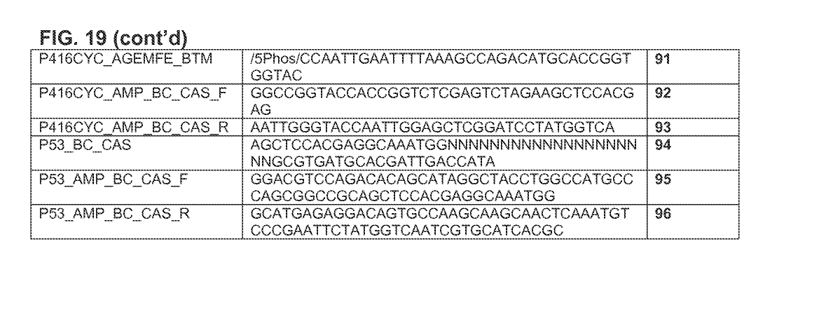

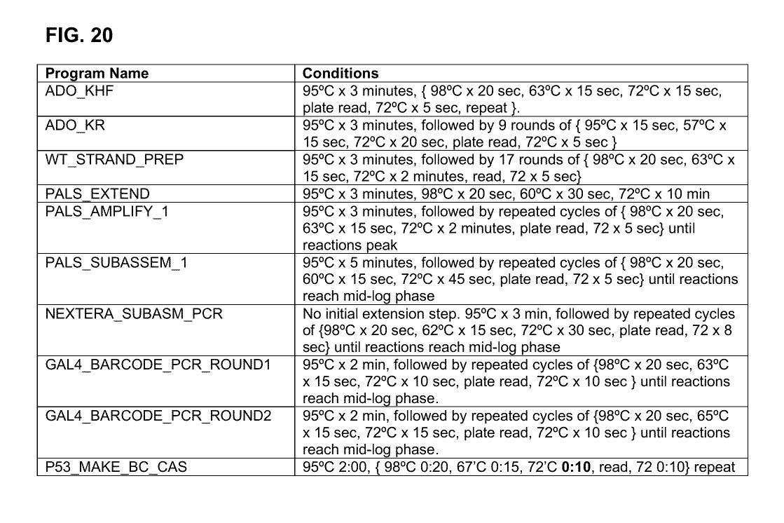

Each primer was designed as a 90mer, including flanking 15-base flanking adaptor sequences (i.e., 5′ flanking adaptor sequence and 3′ flanking adaptor sequence), except for the Gal4 in-frame codon deletion primers, which were designed as 87mers. Each primer is synthesized sense to the gene, with 33 upstream bases, followed by the codon replacement, and 24 downstream bases. To allow for specific retrieval, a different flanking adaptor pair was used for each subset of mutagenic primers on the array. Gal4 primers were flanked by adaptor sequences “truncL_GAL4DBD” and “truncR_GAL4DBD” (see FIG. 19) and p53 primers were flanked by “truncL_TP53” and “truncR_TP53” (see FIG. 19). Mutagenic primer libraries were retrieved by PCR using the respective adaptor pair (“L_TP53”/“R_TP53” or “L_GAL4DBD”/“R_GAL4DBD”) (see FIG. 19), using 10 ng of the starting oligo pool as template using Kapa Hifi Hot Start ReadyMix (“KHF HS RM”, Kapa Biosystems) and following the cycling program “ADO_KHF” (see FIG. 20). Reactions were monitored by fluorescent signal on a BioRad Mini Opticon real-time thermocycler, and were removed after 15 cycles. Amplification products were purified with Zymo Clean & Concentrate 5 columns (Zymo Research). Electrophoresis on a 6% TAE polyacrylamide gel confirmed a single band of ˜108 bp for each library, corresponding to the original oligo size plus 18 bp of additional adaptor sequence added by PCR (FIG. 11).

The resulting oligo pools were further amplified with adaptors modified to contain a deoxyuracil base at the 3′ terminus. This second-round amplification was carried out in 50 ul reactions, using 1 ul of the previous amplification reaction (at a 1:4 dilution in dH2O) as template, following cycling program “ADO_KR”. Each reaction included 25 ul Kapa Robust Hot Start ReadyMix (which is not inhibited by uracil-containing templates), amplification primers at 500 nM each (“L_“GAL4DBD”/“R_GAL4DBD_U” or “L_TP53”/“R_TP53_U”) (see FIG. 19), and SYBR Green I at 0.5×. Immediately following PCR, each library was denatured at 95° C. for 30 seconds, and then snap cooled on ice. To cleave the “R” adaptors, 2 U USER enzyme mix (New England Biolabs) was added, and each reaction was incubated for 15 minutes at 37° C. Finally each reaction was supplemented by 2.5 ul of a 10 uM stock of the corresponding “L” primer (“L_GAL4DBD” or “L_TP53”) (see FIG. 19), followed by one final cycle of annealing/priming/extension. Amplification products were purified as before on Zymo columns. Gel electrophoresis confirmed that each resulting library was a mixture of off-product flanked on both sides by adaptors (108 bp), and the desired product with only “L” adaptors (84 bp, FIG. 11).

Wild-Type Template Preparation.

The full-length Gal4 open reading frame was amplified from genomic DNA of S. cerevisae strain BY4741 and directionally cloned into the yeast shuttle vector p416CYC, a single-copy CEN plasmid with the CYC1 promoter [35], by digestion with SmaI and ClaI (New England Biolabs), using the InFusion cloning kit (Clontech). Subsequently, an N-terminal truncation was prepared by amplifying residues 1-196 from the original clone using the primer pairs GAL4_CLONE_F and GAL4_NTERM_R (see FIG. 19), and recloning into p416CYC to create p416CYC-Gal4Wt-1-196. For p53, a wild-type clone with a Cterminal GFP fusion was purchased from OriGene (#RG200003).

To prepare wild-type sense and antisense strands to serve as templates for mutagenic primer extension, the desired fragments were amplified from plasmid clones by PCR with several modifications. To select for the sense strand, the reverse primer was phosphorylated to allow for its later degradation by lambda exonuclease, and to select the antisense strand, the forward primer was instead phosphorylated. Furthermore, to minimize undesired carry-through of wild-type copies, in some cases long synthetic tails (38 or 40 nt) were placed on the phosphorylated primer to prevent the resulting 3′ ends of the selected strands from acting as primers during subsequent extension steps. Primers were either ordered with a 5′ phosphate or were enzymatically phosphorylated in 10 ul reactions containing 1 ul of 100 uM primer stock, 7 ul H2O, 1 ul 10× T4 Ligase Buffer with ATP (NEB), and 10 U T4 polynucleotide kinase (NEB) and incubated for 30 minutes at 37° C., followed by heat inactivation for 20 minutes at 65° C. and one minute at 95° C. Wild-type fragments were amplified in 50 ul PCR reactions with forward and phosphorylated reverse primers using Kapa HiFi U+ HotStart Ready Mix (“KHF U+ HS RM”) supplemented with dUTPs to a final concentration of 200 nM. Primers for wild-type template preparation are listed in FIG. 19, and amplification used cycling conditions “WT_STRAND_PREP” (see FIG. 20). For starting template, 200 pg of each wild-type clone plasmid was used. Amplification products were purified by Zymo column, and to select the desired strand, 30 ng of each PCR product was treated for 30 min at 37° C. with 7.5 U lambda exonuclease (NEB) in a 30 ul reaction containing lambda exonuclease buffer at 1× final. Reactions were heat killed for 15 minutes at 75° C. and purified by Zymo column (5 volumes binding buffer, eluted in 10 ul buffer EB).

Mutagenic Primer Extension.

Next, 2 ng of each primer pool was combined with 3 ng of its respective sense-strand template, raised to 12.5 ul with dH2O, and mixed with 12.5 ul of KHF U+HS RM for extension along the dUTP-containing wild-type template by the annealed mutagenic primers. The reaction was subjected to one round of denaturation, annealing, and extension (cycling conditions “PALS_EXTEND”; see FIG. 20), purified by Zymo column, treated with 1.5 U USER enzyme for 10 minutes at 37° C. to degrade the wild-type template, and purified again by Zymo column (same conditions).

The resulting strand extension products were enriched via PCR using the KHF U+ HS RM in 25 ul reactions using the cycling program PALS_AMPLIFY (see FIG. 20) and 3 ul of preceding strand extension product as template. Reactions were monitored by SYBR Green fluorescence intensity and removed in mid-log phase (13 cycles for Gal4, 10 cycles for p53). The forward and reverse primers corresponding to the sense strand template and the mutagenic adaptor, respectively, were “OUTER_F”/“GAL4DBD_U” (for Gal4; see FIG. 19) or “P53_SENSE_F”/“L_TP53_U” (for p53; see FIG. 19). An aliquot of each amplification product was visualized by PAGE electrophoresis, and appeared as a smear over the desired size ranges (˜450-650 bp for Gal4, ˜300-1500 bp for p53) (FIG. 11.).

The reverse primer in the preceding amplification step carried a 3′-terminal dUTP, allowing for adaptor excision by treatment with 1 U USER enzyme for 15 minutes at 37° C. This reaction was cleaned by Zymo column and eluted in 11.8 ul buffer EB. Next, the respective forward primer was added (0.75 ul at 10 uM) followed by 12.5 ul of KHF HS RM to create sense-strand mutagenized megaprimers with one round of cycling conditions “PALS_EXTEND” (see FIG. 20). For this step, the non-uracil tolerant PCR mastermix was used to limit amplification of any remaining uracil-containing wildtype strand template.

Sense-strand megaprimers were then purified by Zymo column, annealed to the wildtype antisense strand, and extended to form full length copies. Each extension reaction contained 3 ng of the sense-stranded megaprimer pool, 1 ng of the wild-type dUTP-containing antisense strand, and was performed with KHF U+ HS RM, followed by column cleanup, USER treatment (1.5 U for 10 min at 37° C.), and a second column cleanup, as during the initial mutagenic strand extension reaction. Finally, the full-length mutagenized copies were enriched by PCR using fully external primers (“OUTER_F”/“GAL4_OUTER_R” or “OUTER_F”/“P53_ANTISENSE_R”) (see FIG. 19), in 25 ul PCR reactions with KHF U+ HS RM with conditions “PALS_AMPLIFY” (see FIG. 20).

PALS Library Cloning.

Gal4 DBD PALS libraries were cloned into p416CYC-bc, a pre-tagged library of vectors derived from p416CYC, in which each clone contains a random 16mer tag. To prepare p416CYC-bc, a pair of unique restriction sites was placed downstream of the CYC1 terminator by digesting p416CYC with KpnI-HF (NEB) and inserting a duplex of oligos (“P416CYC_AGEMFE_TOP”/“P416CYC_AGEMFE_BTM”) (see FIG. 19) by ligation to create the following series of restriction sites: KpnI-AgeI-MfeI-KpnI. A tag cassette containing a randomized 16mer (“P416CYC_BC_CAS”) (see FIG. 19) was then PCR-amplified using primers “P416CYC_AMP_BC_CAS_F”/“P416CYC_AMP_BC_CAS_R” (see FIG. 19) and cycling program “MAKE_BC_CAS” (see FIG. 20), to add priming sites for later tag counting during Gal4 functional selections, and to add flanking AgeI and MfeI sites. The resulting tag cassette amplicon was directionally cloned into the modified p416CYC vector by double-digestion with AgeI-HF and MfeI-HF (NEB) and transformed into ElectroMax DH10B electrocompetent E. coli (Invitrogen), to yield ˜9.2×106 distinctly tagged clones. The resulting library, p416CYC-bc, was expanded by bulk outgrowth and purified by midiprep using the ChargeSwitch Pro Midi kit (Invitrogen). Next, 15 ug of p416CYC-bc was digested with 40 U SmaI (NEB) for 1 hr at 25° C. in 60 ul, followed by addition of 20 U ClaI (NEB), digestion for 1 hr at 37° C., and purification by MinElute column (Qiagen). To insert the Gal4 DBD PALS library, 50 ng of the final PALS PCR product was combined with 10 ng SmaI/ClaI linearized p416CYC-bc vector and directionally cloned using the InFusion HD kit (ClonTech), as directed. Libraries were transformed by electroporation into 10-beta electrocompetent E. coli (NEB), and bulk transformation cultures were expanded overnight in 25 ml LB+ampicillin (50 ug/ml) at 37° C., shaking at 250 rpm. Due to the large number of vector copies present in the cloning reaction, pairing of Gal4 mutant inserts with barcodes is essentially sampling with replacement; the number of positive clones (˜9.0×105) is less than the number of tags by approximately an order of magnitude, so only ˜0.45% of tags are estimated to be paired with two different inserts.

Tagged p53 PALS libraries were created in the reverse order: the PALS-mutagenized amplicon was cloned first, and the library was expanded and tags inserted second. The p53 library was cloned into pCMV6-AC-GFP (Origene) by standard directional cloning in two separate cloning reactions using NotI-HF/BamHI-HF or NotI-HF/KpnI-HF (NEB). Libraries were transformed into 10-beta electrocompetent cells (NEB), combined, expanded overnight and purified by midiprep as for Gal4. Subsequently, the cloned p53 libraries were linearized at the AgeI site downstream of the hGH poly-A signal: 2.5 ug of plasmid DNA was digested with 10 U AgeI (NEB) in 50 ul for 1 hr at 37° C., and purified by Zymo column. A tag cassette containing a randomized 20mer was synthesized (“P53_BC_CAS”) (see FIG. 19) and PCR amplified for cloning (using primers “P53_AMP_BC_CAS_F”/“P53_AMP_BC_CAS_R”) (see FIG. 19), using KHF RM HS and cycling program “MAKE_BC_CAS” (see FIG. 20). Tags were directionally inserted at the AgeI site by InFusion cloning, as for Gal4, and the resulting plasmid was transformed, expanded in bulk, and purified by midiprep as in the first round of cloning.

Clone Subassembly Sequencing.

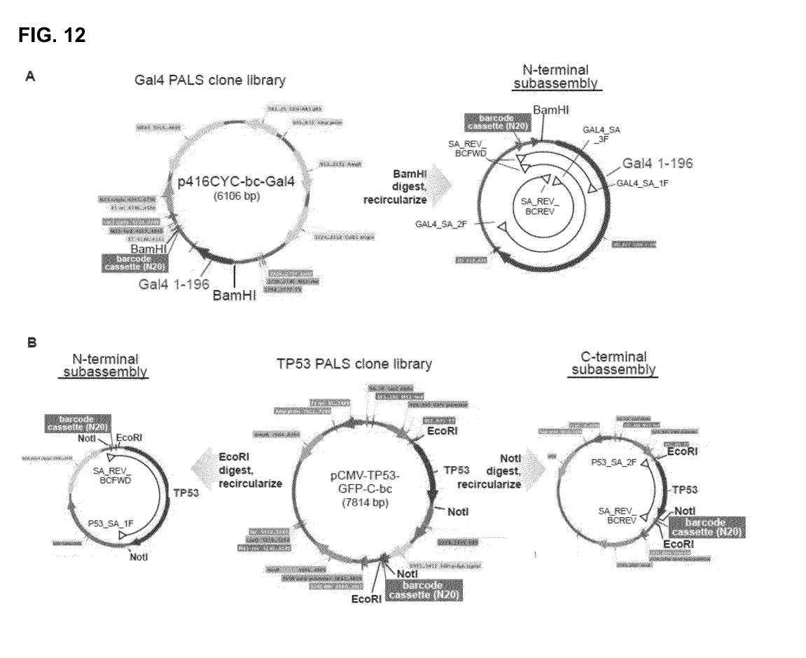

To bring the tag cassette into proximity with the mutagenized Gal4 coding sequence (FIG. 12), 1 ug of the mutant Gal4 plasmid library was digested with 20 U BamHI-HF (NEB) in 1× CutSmart Buffer for 30 minutes at 37° C. The digest was cleaned up by Zymo column, and 200 ng of the product was recircularized by intramolecular sticky-end ligation using 1600 U T4 DNA ligase (NEB) in a 200 ul reaction for 2 hours at 20° C. Following Zymo column cleanup, linear fragments and concatamers were depleted by treatment with 5 U plasmid-safe DNase (Epicentre) for 30 minutes at 37° C., and then 30 minutes at 70° C. Next, PCR was used to amplify fragments containing the tag cassette at one end, and the mutagenized insert, using 3 ul of the heat-killed recircularization product as template (desired recircularization product and primer pairs shown in FIG. 12A) and following cycling conditions “PALS_SUBASSEM” (see FIG. 20). Amplification products were purified using Ampure XP beads (1.5× volumes bead/buffer). P53 PALS clone libraries were recircularized following a similar strategy, except that digestions with EcoRI or NotI followed by recircularization were used individually to bring the tag cassette into proximity with the N or C termini, respectively (FIG. 12B).

To prepare Illumina sequencer-ready subassembly libraries, tag-linked amplicons from the previous step were fragmented and adaptor-ligated using the Nextera v2 library preparation kit (Illumina), with the following modifications to the manufacturer's directions: for each reaction, 1.0 ul Tn5 enzyme “TDE” was combined with 2.0 ul H2O, 5 ul Buffer 2× TD, and 2 ul of the post-recircularization PCR product. Longer insert sizes were obtained by diluting enzyme TDE up to 1:10 in 1× Buffer TD (a 1:4 dilution was used for the libraries sequenced here). Tagmentation was carried out by incubating for 10 minutes at 55° C., followed by library enrichment PCR to add Illumina flowcell sequences. Libraries were amplified by KHF RM 2× mastermix in 25 ul using a forward primer of NEXV2_AD1 and one of the indexed reverse primers “SHARED_BC_REV_###” (see FIG. 19). PCR reactions were assembled on ice using as template 2 ul of the transposition reaction (without purification), and cycling omitted the initial strand displacement step typically used with the Nextera kit (conditions “NEXTERA_SUBASM_PCR”) (see FIG. 20). Lastly, fixed-position amplicon sequencing libraries starting from the mutagenized insert end of the clone were prepared by adding Illumina flowcell adaptors directly to the tag-insert amplicons by PCR, using the same PCR conditions but substituting the forward primer “ILMN_P5_SA” (see FIG. 19) for the Nextera-specific forward primer.

Tag-Directed Clone Subassembly.

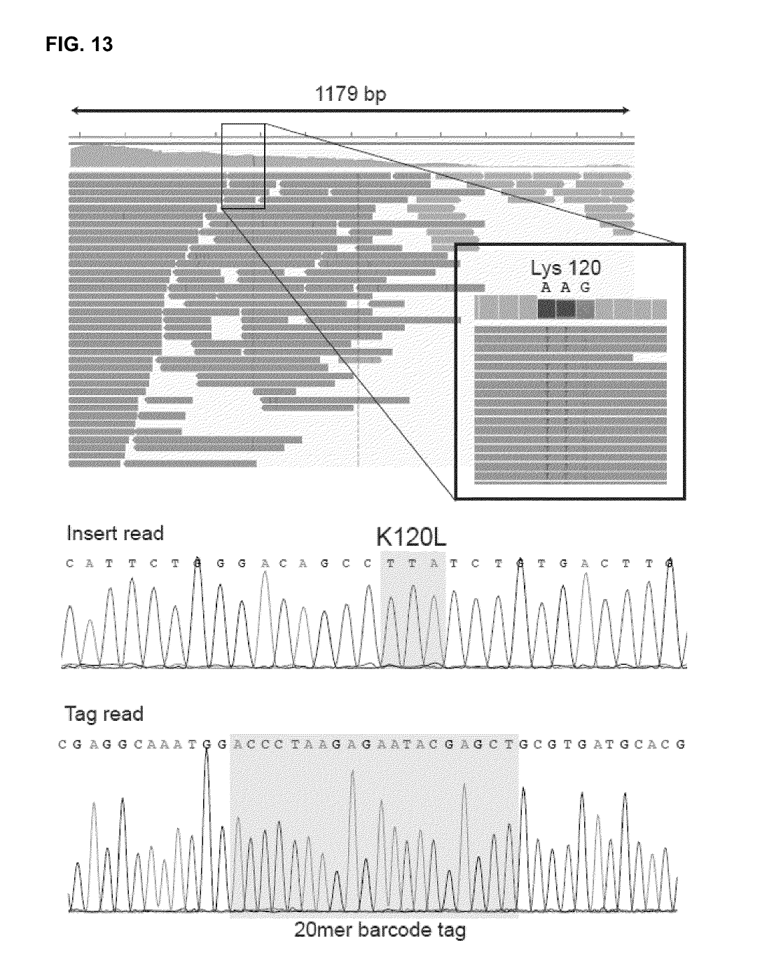

Subassembly libraries were pooled and subjected to paired-end sequencing on Illumina MiSeq and HiSeq instruments, with a long forward read directed into the clone insert (101 bp for HiSeq runs, 325 or 375 bp for MiSeq runs) and a reverse read into the clone tag. Tag-flanking adaptor sequences were trimmed using cutadapt (obtained from https://code.google.com/p/cutadapt/), and read pairs without recognizable tag-flanking adaptors were excluded from further analysis. Insert-end reads were aligned to the Gal4 or p53 wild-type clone sequence using bwa mem (with arguments “-z 1 -M”) [36], and alignments were sorted and grouped by their corresponding clone tag. To properly align the programmed in-frame codon deletions included in the Gal4 PALS library, bwa alignments were realigned using a custom implementation of Needleman-Wunsch global alignment with a reduced gap opening penalty at codon start positions (match score=1, mismatch score=−1, gap open in coding frame=−2, gap open elsewhere=−3, gap extend=−1). A consensus haplotype sequence was determined for each tag-defined read group by incorporating variants present in the group's aligned reads at sufficient depth. Spurious mutations created by sequencing errors, or mutations present at low allele frequency arising from linking two haplotypes to the same tag were flagged and discarded by requiring the major allele at each position (either wild-type or mutant) to be present with a frequency of ≧80%, ≧75% and ≧66%, for read depths≧20, 10-19, or 3-10, respectively, considering only bases with quality score≧20. Tag groups with fewer than three reads (Gal4 DBD) or 20 reads (p53) were discarded, as were groups not meeting the major allele frequency threshold across the entire target (Gal4 DBD) or a minimum of 1 kbp (p53). Consensus haplotypes were validated by Sanger sequencing of individual colonies from each tagged plasmid library (FIG. 13).

Gal4 Functional Selections.

Gal4 DBD PALS libraries were transformed into chemically competent S. cerevisae strain PJ69-4alpha prepared using a modified LiAc-PEG protocol, as previously described [9, 37]. After transformation, cells were allowed to recover for 80 minutes at 30° C. shaking at 250 rpm. To select for transformants, cultures were spun down at 2000×g for 3 min, resuspended and grown overnight at 30° C. in 40 ml SC media lacking uracil. Plating 0.25% of the recovery culture prior to outgrowth indicated a library titer of ˜2≦105 transformants. Following overnight outgrowth, glycerol stocks were prepared from the transformation culture and stored at −80° C.

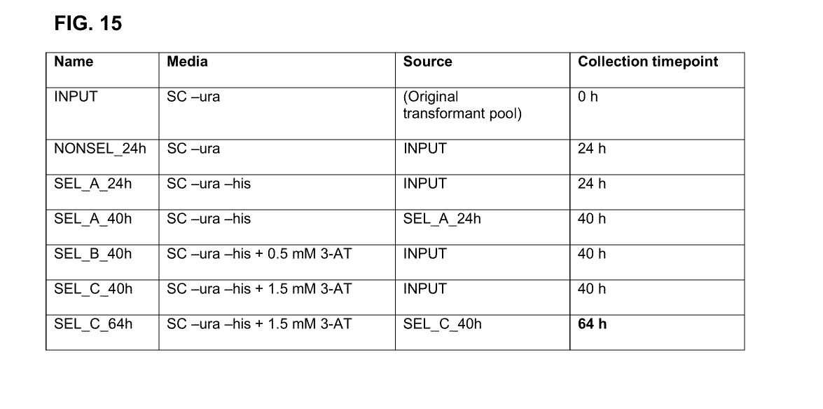

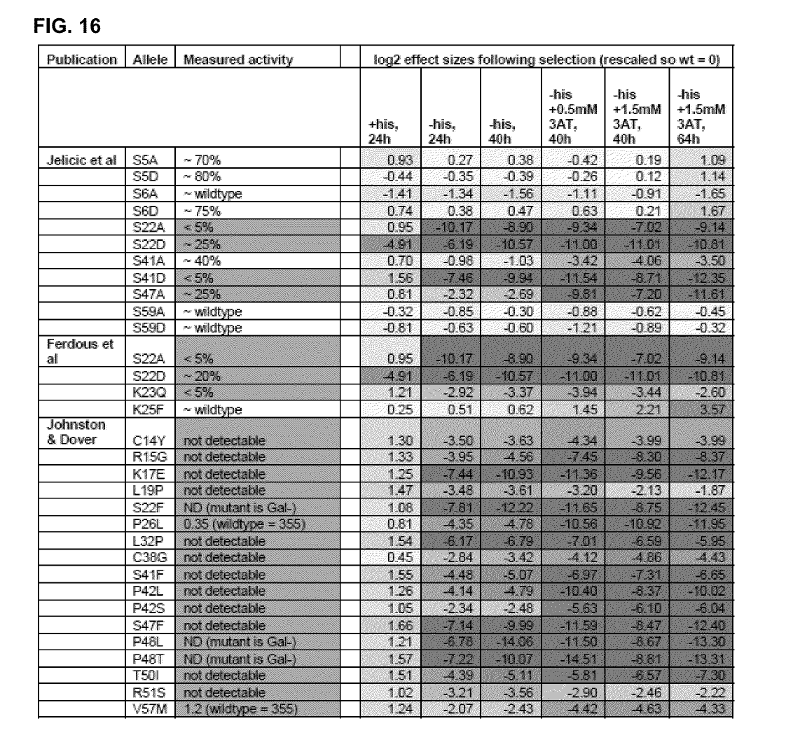

Frozen stocks of yeast carrying the Gal4 DBD PALS library were thawed and recovered overnight in 50 ml SC media lacking uracil. An aliquot of 1 ml (˜1.8×106 cells) was pelleted and frozen as the baseline input sample, and equal aliquots were used to inoculate each of four 40 ml cultures of SC media either lacking uracil (nonselective) or lacking both uracil and histidine and optionally containing the competitive inhibitor 3-AT (selective, FIG. 15). Cultures were maintained at 30° C. and checked at 24 h, 40 h, and 64 h. After reaching log-phase (OD 600>=0.5), each culture was serially passaged by inoculating 1 ml into 40 ml fresh media.

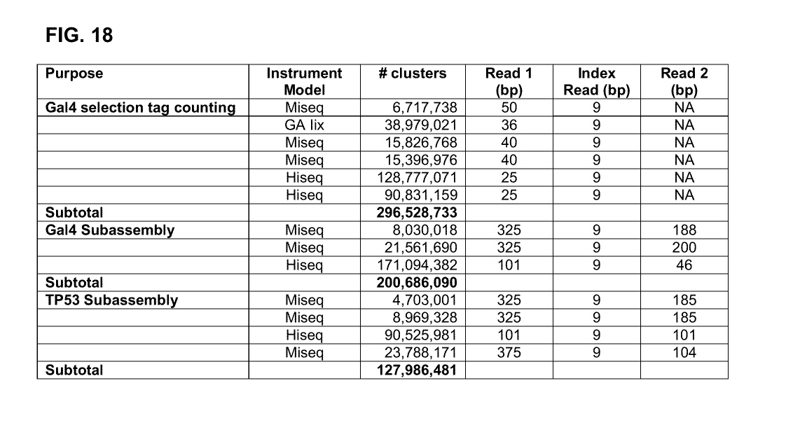

Input and post-selection cultures were pelleted at 16000×g and frozen at −20° C. Gal4 plasmids were recovered by spheroplast preparation and alkaline lysis miniprep using the Yeast Plasmid Miniprep II kit as directed (Zymo Research). Two-stage PCR was then used to amplify and prepare sequencing libraries to count the plasmid-tagging tags. In the first step, 2.5 ul of miniprep product was used as template in 25 ul reactions with KHF RM HS, with primers flanking the tag cassette (“GAL4_BC_AMP_F”/“GAL4_BC_AMP_R”) (see FIG. 19), using the program “GAL4_BARCODE_PCR_ROUND1” (see FIG. 20) for 15-17 cycles. The resulting product was used directly as template (1 ul, without cleanup) for the second-stage PCR reaction to add Illumina flowcell-compatible adaptors as well as sample-indexing barcodes to allow pooled sequencing (forward primer “GAL4_ILMN_P5”, and reverse primer one of “SHARED_BC_REV###” (see FIG. 19)). For the second round, the cycling program “GAL4_BARCODE_PCR_ROUND2” (see FIG. 20) was followed for 5-7 cycles. Tag libraries were cleaned up with AmpPure XP beads (2 volumes beads+buffer) and were sequenced across several runs on Illumina MiSeq, GAIIx, and HiSeq instruments (FIG. 18), using 25-50 bp reads.

Gal4 Enrichment Scores.

Tag reads were demultiplexed to the corresponding sample using a 9 bp index read, allowing for up to two mismatches. Tag reads lacking the proper flanking sequences or containing ambiguous ‘N’ base calls were discarded, and per-barcode histograms were prepared by counting the number of occurrences of each of the remaining tags. Tags were required to exactly match the tag of a single subassembled haplotype, and were then normalized to account for differing coverage over each library by dividing by the sum of tag counts.

An effect score was calculated for each amino acid mutation by summing the read counts of tags corresponding to all the subassembled clones carrying that mutation as a singleton, divided by the equivalent sum for wild-type clones, and taking a log-ratio between the selection and input samples, as shown in Equation 1 below:

$${e}_{MUTi} = {log}_{2} ( \frac{{\sum }_{TAG j ∈ MUTi} {r}_{SEL , j} + 1}{{\sum }_{TAG k ∈ MUTi} {r}_{SEL , k} + 1} ) - {log}_{2} ( \frac{{\sum }_{TAG j ∈ MUTi} {r}_{INPUT , j} + 1}{{\sum }_{TAG k ∈ MUTi} {r}_{INPUT , k} + 1} )$$

where r_{SEL,j} and r_{INPUT,j} are the read counts of tag j in the selected and input samples, respectively.

Evolutionarily conserved residues in Zn2/Cys6 domains were identified by querying HHblits with Gal4 residues 1-70 [38], and were displayed using Weblogo [39]. To compare core and outward-facing residues within the dimerization helix, residues 51-65 were each scored for distance to the overall structure's solvent-exposed surface predicted using MSMS43 (using the Gal4(1-100) crystal structure, PDB accession 3COQ). Residues with above-median distance to the surface were considered ‘core’, and those with below-median distance were considered ‘exposed’, and the log 2E values of the two subsets were compared by the Mann-Whitney U test.

Gal4 Validations.

For qualitative validation of Gal4 missense mutation effects, specific alleles (C14Y, K17E, K25W, K25P, L32P, K43P, K45I, and V57M) were individually introduced into p416CYC-Gal4Wt-1-196 using the Quickchange mutatgenesis kit (Agilent) following the manufacturer's directions. Mutant colonies were miniprepped and verified by capillary sequencing, and transformed into PJ69-4alpha by LiAc treatment. Following transformation, a single yeast colony transformed by mutant or wild-type Gal4 constructs was picked and expanded in overnight culture, and back-diluted to OD 0.2 and allowed to return to mid-log phase before spotting ten-fold dilutions starting with an equal number of cells onto nonselective plates (SC lacking uracil) or selective plates (SC lacking uracil and histidine, supplemented with 5 mM 3-AT).

Results

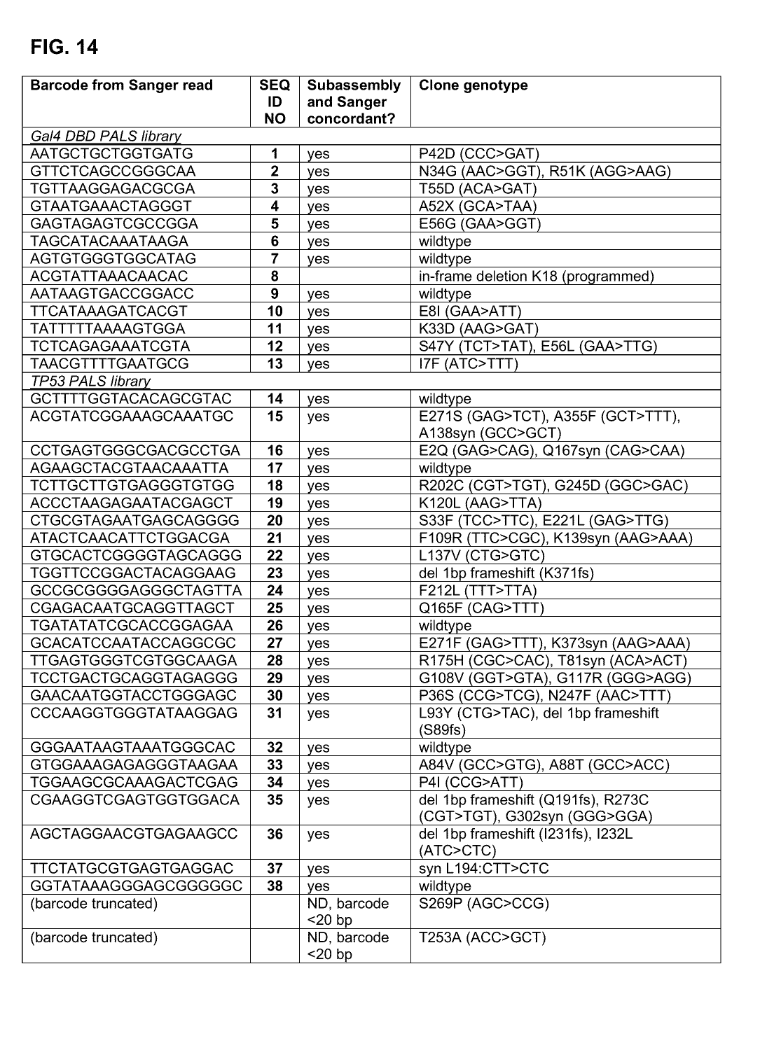

As a proof-of-principle, first a PALS library for the DNA-binding domain (DBD) of Gal4, an archetypal yeast transcription factor, was constructed. Each codon of the DBD (residues 2-65) for replacement was targeted either by the yeast-optimized codon for each of the 19 other amino acids, or by a premature STOP. The Gal4 PALS library was cloned into a yeast expression vector, followed by tagging and subassembly requiring a minimum coverage of three reads at each nucleotide across the entire cloned ORF. Of the resulting sequence-verified consensus haplotypes, ˜47% carried one and only one programmed mutation on an otherwise wild-type background (Table 1). Among these “clean” clones, 99.9% (n=1,342) of the programmed single-codon replacements were observed at least once and 99.8% were observed at least five times. In addition, the ability of PALS to program more complex mutations was investigated by including a tiling set of in-frame deletion variants targeting each codon and found all single codon deletions within the resulting library. To validate the accuracy of these subassemblies, 40 clones were randomly picked and performed capillary sequencing on the mutagenized gene insert and its accompanying tag, confirming the subassembly-derived haplotype without any additional mutations (FIG. 14).

To assess the scalability of this approach to full-length human genes, PALS mutagenesis was performed on the entire coding sequence of the human tumor suppressor p53. In contrast to Gal4, for which each mutant codon was explicitly specified, p53 codons were targeted for replacement by degenerate (“NNN”) triplets, reducing the number of required microarray features to the total number of codons (393 for p53) and allowing access to synonymous variants. Given its greater length, there was greater potential for incidental secondary mutations in p53 due to PCR error or chimerism. Accordingly, a lower rate of sequence-verified single-mutant haplotypes than for Gal4 (27%, n=177,841) was observed. Despite the lower purity of this library and despite sequencing fewer clones per residue, 93.4% (n—7,345) of the desired amino acid substitutions were still observed as clean, single-mutant clones.

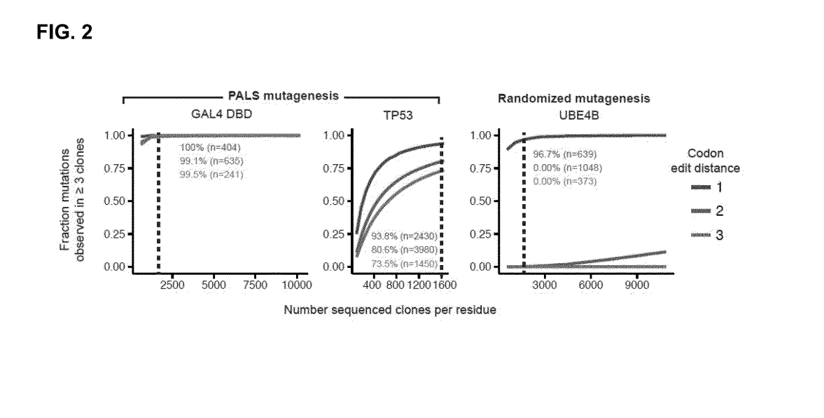

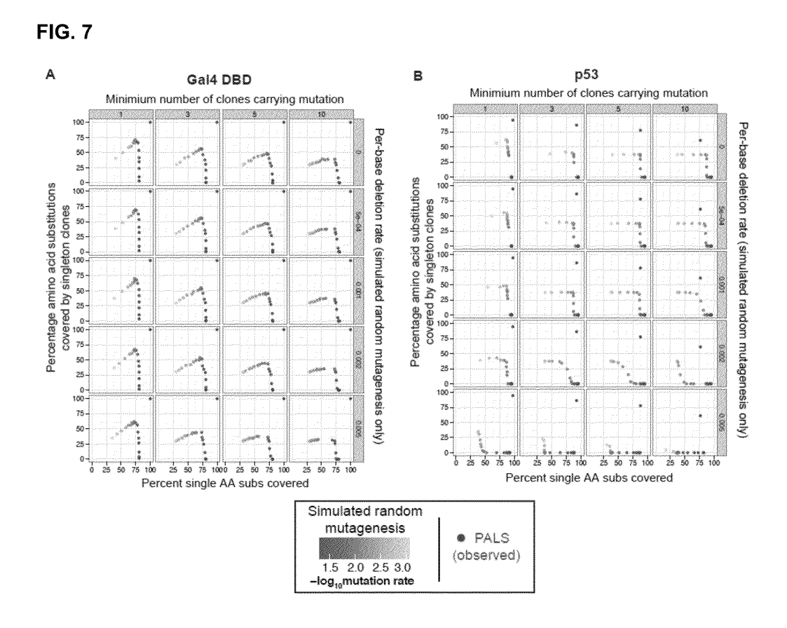

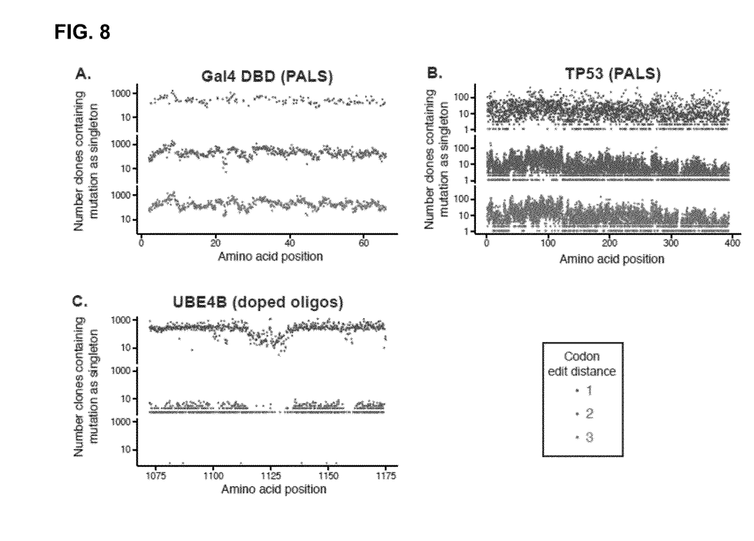

The uniformity and coverage of mutations introduced by PALS was examined using these full-length clone sequences. For comparison of performance, a random mutagenesis library constructed by randomized doped oligonucleotide synthesis of a 102 amino-acid fragment of the mouse E3 ubiquitin ligase gene Ube4b [8] was concurrently analyzed. This library comprised 1.12 million full-length clone sequences, of which 16.6% contained a single codon mutation. Codon substitutions requiring 2-bp and 3-bp changes, which were abundantly represented within PALS libraries, were almost entirely absent from the random mutagenesis library at a comparable depth of coverage from sequenced clones (FIG. 2). Idealized simulations indicate that varying the randomized mutagenesis rate can partly increase coverage of these missing codon substitutions but at the cost of creating many more clones with mutations at multiple residues, including nonsense codons (FIG. 7). Mutational coverage by PALS was relatively uniform across the length of each gene with moderate bias towards the N-terminal half of each gene (1.1-fold for Gal4 DBD; 2.2-fold for p53), possibly reflecting the relative inefficiency of longer strand extensions following initial mutagenic primer annealing (FIG. 8).

To demonstrate the utility of PALS mutagenesis for deep mutational scanning, the Gal4 DBD library was subjected to selection for its ability to transcriptionally activate a yeast reporter gene. The mutagenized Gal4 DBD library was cloned into a low-level expression vector along with the wild-type sequence encoding an additional 131 amino acids. The resulting 196 amino-acid N-terminal fragment retains the same DNA-binding specificity as full-length Gal4, and is sufficient for transcriptional activation [17], but it lacks the cellular toxicity due to expression of full-length Gal4 which is likely caused by sequestration of the transcriptional machinery [18]. The Gal4(1-196) PALS library was transformed into the yeast two-hybrid reporter strain PJ69-4alpha [19], which is deleted for GAL4 and has expression of the HIS3 gene under the control of the GAL1 promoter. Thus, growth of yeast on media lacking histidine was conditional upon the ability of the introduced Gal4 allele to bind to and activate HIS3. Selection stringency was modulated by addition of 3-amino-1,2,4-triazole (3-AT), a competitive inhibitor of His3. After selection for Gal4 function, deep sequencing was performed to quantify the enrichment or depletion of each Gal4 mutant. Rather than resequencing the mixed population of full-length inserts, the frequency of the 16-mer tags, which are individually associated with full-length inserts via subassembly, was sequenced and tabulated.

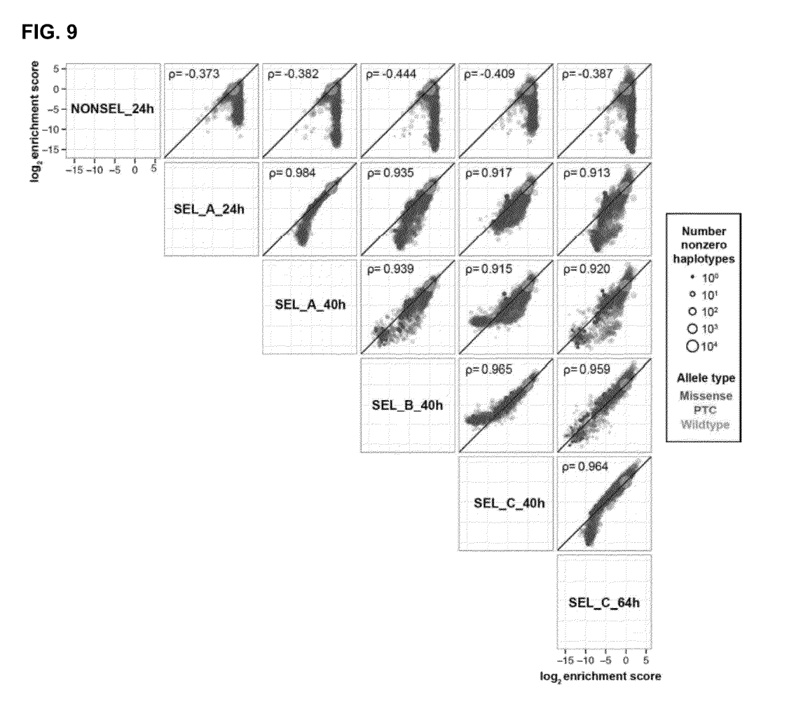

296.5 million tag reads were collected across the input library and six selection time points (FIG. 15). Summing the counts of tags that are associated with identical clones containing single amino acid mutations, per-mutation effect sizes (log 2E) for the 98.4% of mutations (1318/1340) that were each represented by three or more distinct tagged clones in the input library were then calculated. After two rounds of yeast outgrowth under stringent conditions (t=64 h in-histidine media supplemented with 1.5 mM 3-AT), the enrichment score distribution was shifted downward, with 57.3% of single amino acid mutants strongly depleted (log 2E<−3). Premature stop mutations were nearly uniformly deleterious under selective but not permissive conditions (median log 2E=−5.75 and 1.33, respectively). Per-mutation effect sizes were well-correlated between sequential time-points for each selection (Spearman's ρ=0.964-0.984) as well across selections (ρ=0.917-0.965, FIG. 9). Nearly a third of the residues (19-27 of 64, depending on selection time-point) were strongly intolerant to mutation, with their median effect size for non-truncation mutants at least as low as the overall median of premature truncation mutants. Overall, a comparable proportion of codon substitutions (25.9% to 33.9%) were similarly deleterious.

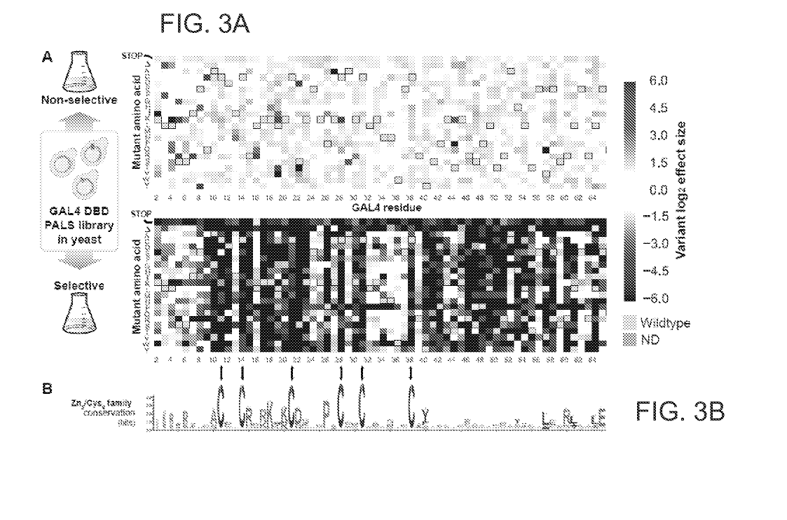

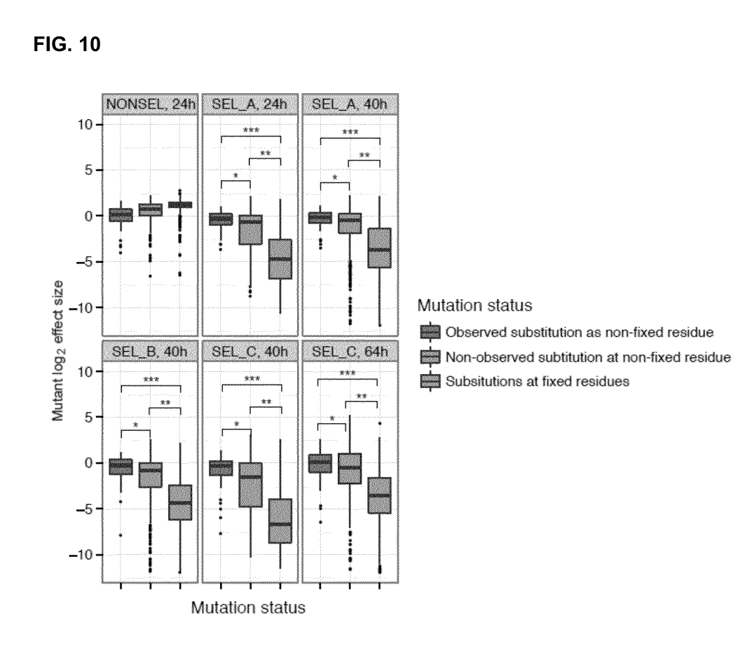

The resulting profile of functional constraint (FIG. 3A) recapitulates many of the hypomorphic and loss-of-function alleles found by initial forward genetic screens [20], and highlights key residues in agreement with structural [21, 22] and biochemical studies [23, 24]. Gal4 binds DNA as a homodimer via a Zn2Cys6-class domain centered on a pair of Zn2+ ions which help to maintain the fold of the DNA-binding residues. The six chelating cysteines are tightly conserved throughout evolution and are critical for Gal4 function (FIG. 3B). Accordingly, they appear among the most intolerant to amino-acid substitution, along with lysines 17 and 18, which contact the DNA bases of the CGG-N11-CGG recognition motif. More broadly, evolutionarily conserved residues in close Gal4 orthologs were significantly less tolerant to substitution during selective outgrowth (P<1.6×10−7 comparing per-residue mean log 2E, Mann-Whitney U, FIG. 10) but not following outgrowth in media containing histidine (P=1). At residues that did have substitutions in orthologous proteins, the evolutionarily “accepted” substitutions were less deleterious to Gal4 function than other mutations at the same sites, even without considering substitutions to proline and premature stop (P<0.011 to P<3.5×10⁻⁴ across time points and replicates, Mann-Whitney U).

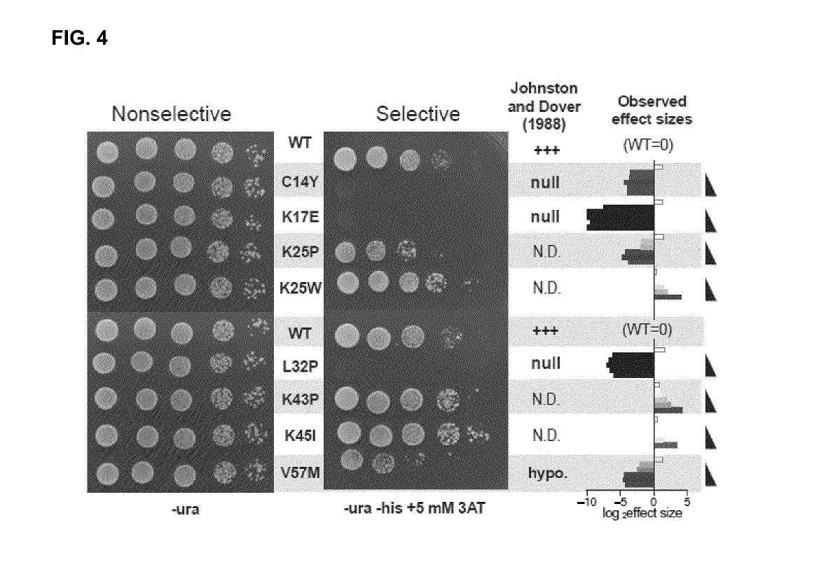

To validate these effect size measurements, eight individual alleles were re-created by conventional site-directed mutagenesis and assayed them for growth defects by a spotting assay (FIG. 4). These included loss-of-function (C14Y, K17E, and L32P) and hypomorphic alleles (V57M) from the initial screens [20], which conferred growth rates in the spotting assay that agreed with their relative depletion during in the deep mutational scan. Likewise, a novel predicted hypomorphic allele (K25P) was validated and confirmed the slight growth advantage conferred by three alleles from the bulk measurements (K25W, K43P, and K45I).

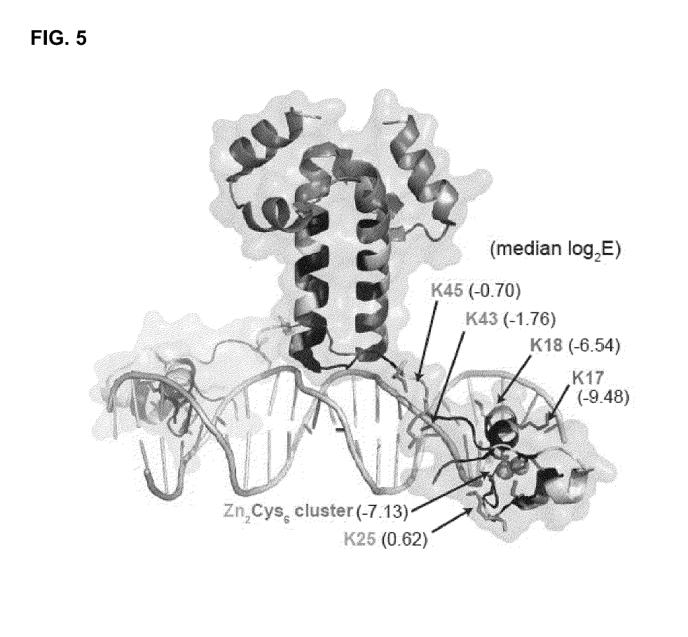

Superimposed on the crystal structure of Gal4 residues 1-100 [22] (FIG. 5), these data highlight several key aspects of Gal4 function. Within the dimerization domain helix (residues 51-65 tested), core residues were on average significantly less tolerant to mutation than outward-facing residues (P<1.6×10⁻⁴ comparing mean log 2E, Mann-Whitney U). A notable exception was E58 (mean log 2E=−6.78), which faces outward but may help to confer specificity to Gal4 dimerization by stabilizing the monomers in the proper register via hydrogen bonding interactions with residues H53 and S47 at the base of helix. Each of these residues was largely intolerant to mutation (mean log 2E=−7.28); however, H53 could be replaced by either a bulky nonpolar tryptophan or polar tyrosine, although it is unclear to what extent these substitutions alter the existing interactions or create new ones. Near the base of the helices, polar, solvent-exposed residues (e.g., T50 and S51) interact with the DNA backbone and were similarly intolerant to substitution, suggesting a role in dimerization or loop positioning.

The linker (residues 41-50) tracks alongside the DNA major groove, making extensive contacts with the negatively charged backbone. A bend at proline 48 aids in positioning the dimerization helix over the DNA minor groove [21], and notably, either of two nearby lysine residues within the linker, K43 and K45, could be mutated to proline without deleterious effects and possibly with a marginal increase in activity (FIG. 4). Throughout most of the rest of Gal4 (other than the disordered N-terminus), proline substitutions were highly deleterious. For instance, leucine 32 is central to one of the two metal-binding domain alpha helices, and showed little constraint overall in the data (mean log 2E=−0.04), aside from replacement with proline completely abrogates Gal4 DNA binding [25]. This trend is broadly observed in deep mutational scans of other proteins, likely reflecting disruption of protein secondary structure due to the proline residue kinking the backbone [26]. Within the Gal4 DBD linker region, however, additional prolines may be beneficial by decreasing the flexibility between the dimerization and zinc-containing regions, making DNA binding and transcriptional activation more entropically favorable. Similarly to most proline mutations, in-frame codon deletions were generally deleterious, with the notable exceptions of K25 and K27, both outward-facing lysines located near proposed sites of post-translational modification in the loop between metal-binding domain helices [23]. Deleterious proline or in-frame deletions at otherwise mutation-tolerant residues (e.g., 32-37) can thus serve to distinguish residues that are structurally important but that do not participate in catalysis or critical post-translational modifications.

### DISCUSSION

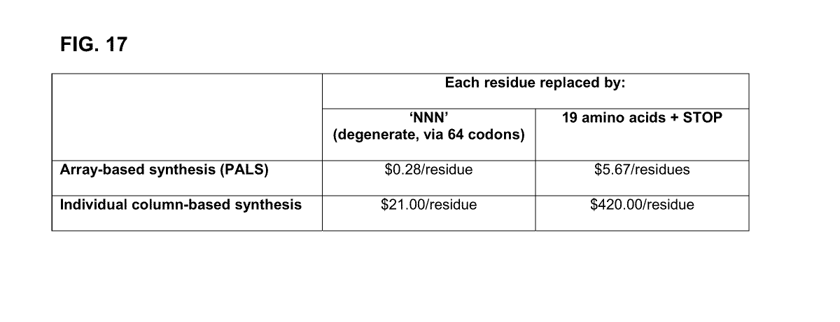

The strategy presented here enables near-comprehensive, single amino acid mutagenesis of a protein-coding sequence in a single reaction, yielding a library that is readily compatible with massively parallel functional analysis. By using primers synthesized in parallel on DNA microarrays, PALS reduces reagent costs by nearly two orders of magnitude compared with previous approaches that require individual oligonucleotide synthesis (FIG. 17), while also markedly reducing labor.

Other functional screens exploiting microarray-derived oligonucleotide libraries have been limited to dense mutagenesis of relatively short sequence elements, due to the length constraints of microarray synthesis (100-200 nt). PALS overcomes this constraint by combining microarray synthesis of short primers with highly multiplexed overlap extension PCR using a wild-type template.

The suitability of PALS libraries for deep mutational scanning was demonstrated by profiling the functional landscape of the Gal4 DBD. PALS provided near complete coverage of codon replacements requiring 2 or 3-bp changes as well as in-frame codon deletions, mutations that would be essentially impossible to obtain at appreciable frequency with randomized mutagenesis strategies. Given its ability to incorporate multibase mutations including indels, PALS could be adapted to other types of screens, for instance to create tiling deletions of long cis-regulatory elements or to recode multiple adjacent codons.

In addition to broadening the scope of sequence variation addressable by large-scale screens, PALS libraries had a lower overall fraction of indel-bearing clones compared to libraries constructed from doped oligonucleotides (13.2%-18.2% versus 28.6%). The resulting improvement in efficiency will be beneficial as deep mutational scanning studies move from being strictly in vitro (e.g., using phage display) into yeast [9] or mammalian tissue culture models. Such studies would ideally use site-specific chromosomal integration, but it remains technically challenging to integrate highly complex libraries, putting a premium on generating as few wasted clones as possible.

There remains room for future improvement towards the goal of ‘pure’ libraries of single-mutant clones. Secondary mutations appear to be dominated by PCR chimera and synthesis errors. These factors were estimated to account for 52% and 24% of the secondary mutations in the Gal4 DBD library, respectively, by counting clones bearing two programmed mutations, or one programmed mutation and secondary mutations within the boundaries of the corresponding mutagenic primer. Chimerism is a technical challenge commonly encountered while amplifying libraries of homologous sequences [31], when incomplete strand extension products in one cycle of amplification act as primers in the subsequent cycle. Future optimization efforts will be directed at quantifying and mitigating this phenomenon by manipulating input template concentration and minimizing amplification cycles, or alternatively using droplet PCR [32]. To reduce the impact of synthesis errors, PALS uses short oligonucleotides (90 nt), but it will nevertheless benefit from ongoing developments in high-fidelity synthesis [33]. In addition, as single-base deletions are the dominant synthesis error mode [34], stringently size-selecting primer libraries may further enrich for primers lacking undesirable secondary mutations. Another strategy would fuse libraries in-frame to a selectable marker in the bacterial cloning host, although the preliminary observations suggest that such selection is inefficient for proteins that do not fold or express well in E. coli.

The combination of PALS mutagenesis and tag-directed subassembly sequencing provides an efficient way to quantify the functional impacts of specific variant alleles within a population of cDNA clones being subjected to multiplex functional analysis. In particular, the sequencing of short tags, each of which is unambiguously associated with a single mutant haplotype, reduces the required sequencing effort, as a short, single-end read contributes a single count for its corresponding mutant haplotype. By contrast, shotgun methods must uniformly cover the entire target gene with reads in order to measure a single count, increasing the cost and introducing additional sampling variability. Sequencing tags rather than inserts also mitigates the impact of sequencing errors, which would otherwise be falsely counted as novel alleles. Even the more accurate sequencing platforms currently available still suffer from considerable error rates near the ends of reads (e.g., the Illumina MiSeq reads used in this study had per-base error rates of ˜2% after 200 bp), necessitating aggressive trimming to avoid encountering sequencing-derived mutations. Emerging long-read sequencing platforms such as Pacific Biosciences or nanopore sequencing may replace subassembly for the pairing of tags with clone inserts, but deep tag counting rather than insert sequencing is likely to remain the most straightforward and accurate method of quantifying effect sizes in deep mutational scans.

PALS mutagenesis holds promise for future deep mutational scans of protein-coding genes, both for basic structure-function studies and for classifying clinically observed alleles as pathogenic or benign, i.e. “pre-measuring” the consequences of variants of uncertain significance before they are observed in a germline or cancer genome. Comprehensively surveying all of codon mutation space, even for replacements that are unlikely to occur naturally, may go beyond identifying key residues to help illuminate potential functional mechanisms or sites of post-translational modification. For proteins that are challenging to crystalize, such as ion channels, structural inferences could be made directly from these scans, supplementing co-evolutionary contact probability models [33]. In sum, this approach—massively parallel synthesis and sequencing coupled to functional selection—provides a general framework to dissect the allelic heterogeneity of human oligogeneic disorders and a path toward functional annotation of the rapidly growing catalogs of variants of unknown significance.

### REFERENCES

The references, patents and published patent applications listed below, and all references cited in the specification above are hereby incorporated by reference in their entirety, as if fully set forth herein.
1. Cunningham, B. C. & Wells, J. A. High-resolution epitope mapping of hGHreceptor interactions by alanine-scanning mutagenesis. Science 244, 1081-1085 (1989).
2. Botstein, D. & Shortle, D. Strategies and applications of in vitro mutagenesis. Science 229, 1193-1201 (1985).
3. Kunkel, T. A. Rapid and efficient site-specific mutagenesis without phenotypic selection. Proc. Natl. Acad. Sci. U.S.A. 82, 488-492 (1985).
4. Weiner, M. P. et al. Site-directed mutagenesis of double-stranded DNA by the polymerase chain reaction. Gene 151, 119-123 (1994).
5. Kato, S. et al. Understanding the function-structure and function-mutation relationships of p53 tumor suppressor protein by high-resolution missense mutation analysis. Proc. Natl. Acad. Sci. U.S.A. 100, 8424-8429 (2003).
6. Araya, C. L. & Fowler, D. M. Deep mutational scanning: assessing protein function on a massive scale. Trends Biotechnol. 29, 435-442 (2011).
7. Fowler, D. M. et al. High-resolution mapping of protein sequence-function relationships. Nat Meth 7, 741-746 (2010).
8. Starita, L. M. et al. Activity-enhancing mutations in an E3 ubiquitin ligase identified by high-throughput mutagenesis. Proceedings of the National Academy of Sciences 110, E1263-72 (2013).
9. Melamed, D., Young, D. L., Gamble, C. E., Miller, C. R. & Fields, S. Deep mutational scanning of an RRM domain of the Saccharomyces cerevisiae poly(A)-binding protein. RNA 19, 1537-1551 (2013).
10. Wong, T. S., Roccatano, D., Zacharias, M. & Schwaneberg, U. A statistical analysis of random mutagenesis methods used for directed protein evolution. J. Mol. Biol. 355, 858-871 (2006).
11. Firnberg, E. & Ostermeier, M. P Funkel: Efficient, Expansive, User-Defined Mutagenesis. PLoS ONE 7, e52031 (2012).
12. Hietpas, R. T., Jensen, J. D. & Bolon, D. N. A. Experimental illumination of a fitness landscape. Proceedings of the National Academy of Sciences 108, 7896-7901 (2011).
13. Roscoe, B. P., Thayer, K. M., Zeldovich, K. B., Fushman, D. & Bolon, D. N. A. Analyses of the Effects of All Ubiquitin Point Mutants on Yeast Growth Rate. J. Mol. Biol. 425, 1363-1377 (2013).
14. Qi, H. et al. A quantitative high-resolution genetic profile rapidly identifies sequence determinants of hepatitis C viral fitness and drug sensitivity. PLoS Pathog 10, e1004064 (2014).
15. Firnberg, E., Labonte, J. W., Gray, J. J. & Ostermeier, M. A Comprehensive, High-Resolution Map of a Gene's Fitness Landscape. Molecular Biology and Evolution (2014). doi:10.1093/molbev/msu081
16. Jain, P. C. & Varadarajan, R. Analytical Biochemistry. Analytical Biochemistry 449, 90-98 (2014).
17. Patwardhan, R. P. et al. High-resolution analysis of DNA regulatory elements by synthetic saturation mutagenesis. Nat Biotechnol 27, 1173-1175 (2009).
18. Hiatt, J. B., Patwardhan, R. P., Turner, E. H., Lee, C. & Shendure, J. Parallel, tag directed assembly of locally derived short sequence reads. Nat Meth 7, 119-122 (2010).
19. Ma, J. & Ptashne, M. Deletion analysis of GAL4 defines two transcriptional activating segments. Cell 48, 847-853 (1987).
20. Gill, G. & Ptashne, M. Negative effect of the transcriptional activator GAL4. Nature 334, 721-724 (1988).
21. James, P., Halladay, J. & Craig, E. A. Genomic libraries and a host strain designed for highly efficient two-hybrid selection in yeast. Genetics 144, 1425-1436 (1996).
22. Johnston, M. & Dover, J. Mutational analysis of the GAL4-encoded transcriptional activator protein of Saccharomyces cerevisiae. Genetics 120, 63-74 (1988).
23. Marmorstein, R., Carey, M., Ptashne, M. & Harrison, S. C. DNA recognition by GAL4: structure of a protein-DNA complex. Nature 356, 408-414 (1992).
24. Hong, M. et al. Structural Basis for Dimerization in DNA Recognition by Gal4. Structure 16, 1019-1026 (2008).
25. Ferdous, A. et al. Phosphorylation of the Gal4 DNA-binding domain is essential for activator mono-ubiquitylation and efficient promoter occupancy. Mol. BioSyst. 4, 1116 (2008).
26. Jeli\check{c}ić, B., Nemet, J., Traven, A. & Sopta, M. Solvent-exposed serines in the Gal4 DNA-binding domain are required for promoter occupancy and transcriptional activation in vivo. FEMS Yeast Res n/a-n/a (2013). doi:10.1111/1567-1364.12106
27. Johnston, M. & Dover, J. Mutations that inactivate a yeast transcriptional regulatory protein cluster in an evolutionarily conserved DNA binding domain. Proc. Natl. Acad. Sci. U.S.A. 84, 2401-2405 (1987).
28. Chou, P. Y. & Fasman, G. D. Empirical predictions of protein conformation. Annu. Rev. Biochem. 47, 251-276 (1978).
29. Melnikov, A. et al. Systematic dissection and optimization of inducible enhancers in human cells using a massively parallel reporter assay. Nat Biotechnol 30, 271-277 (2012).
30. Zhao, W. et al. Massively parallel functional annotation of 3. Nat Biotechnol 32, 387-391 (2014).
31. Lahr, D. J. G. & Katz, L. A. Reducing the impact of PCR-mediated recombination in molecular evolution and environmental studies using a new-generation high fidelity DNA polymerase. Bio Techniques 47, 857-866 (2009).
32. Williams, R. et al. Amplification of complex gene libraries by emulsion PCR. Nat Meth 3, 545-550 (2006).
33. LeProust, E. M. et al. Synthesis of high-quality libraries of long (150mer) oligonucleotides by a novel depurination controlled process. Nucleic Acids Res. 38, 2522-2540 (2010).
34. Kosuri, S. & Church, G. M. Large-scale de novo DNA synthesis: technologies and applications. Nat Meth 11, 499-507 (2014).
35. Kamisetty, H., Ovchinnikov, S. & Baker, D. Assessing the utility of coevolution-based residue-residue contact predictions in a sequence- and structure-rich era. Proceedings of the National Academy of Sciences 110, 15674-15679 (2013).
36. Maurer, K. et al. Electrochemically generated acid and its containment to 100 micron reaction areas for the production of DNA microarrays. PLoS ONE 1, e34 (2006).
37. Nakamura, Y., Gojobori, T. & Ikemura, T. Codon usage tabulated from international DNA sequence databases: status for the year 2000. Nucleic Acids Res. 28, 292 (2000).
38. Mumberg, D., Müller, R. & Funk, M. Yeast vectors for the controlled expression of heterologous proteins in different genetic backgrounds. Gene 156, 119-122 (1995).
39. Li, H. Aligning sequence reads, clone sequences and assembly contigs with BWA-MEM. arXiv (2013).
40. Gietz, R. D. & Woods, R. A. Transformation of yeast by lithium acetate/singlestranded carrier DNA/polyethylene glycol method. Meth. Enzymol. 350, 87-96 (2002).
41. Remmert, M., Biegert, A., Hauser, A. & Söding, J. HHblits: lightning-fast iterative protein sequence searching by HMM-HMM alignment. Nat Meth 9, 173-175 (2012).
42. Crooks, G. E., Hon, G., Chandonia, J.-M. & Brenner, S. E. WebLogo: a sequence logo generator. Genome Research 14, 1188-1190 (2004).
43. Sanner, M. F., Olson, A. J. & Spehner, J. C. Reduced surface: an efficient way to compute molecular surfaces. Biopolymers 38, 305-320 (1996).

What is claimed is:

1. A method for multiplexed mutagenesis of a target nucleotide sequence comprising:
a) generating, in parallel, a set of mutagenic oligonucleotide primers designed to cover all or part of the target nucleotide sequence, wherein each member of the set:
is at least substantially complementary to a portion of the target nucleotide sequence, wherein the portion of the target nucleotide sequence is different for each member of the set,
comprises a 5′ flanking adaptor sequence, a 3′ flanking adaptor sequence, or both, and
comprises a unique programmed mutation near its center; and
b) reacting the set of mutagenic oligonucleotide primers with the target sequence in the presence of a polymerase to generate a mutant nucleotide sequence library, wherein each member of the mutant nucleotide sequence library comprises a full-length copy of the target nucleotide sequence having a unique programmed mutation derived from one member of the set of mutagenic oligonucleotide primers.

2. The method of claim 1, wherein generating the set of mutagenic oligonucleotide primers comprises steps of
synthesizing and releasing a set of mutagenic oligonucleotides from a microarray; and
amplifying and retrieving the set of mutagenic oligonucleotides using the 5′ flanking adaptor sequence or the 3′ flanking adaptor sequence.

3. The method of claim 1, wherein the set of mutagenic oligonucleotide primers are designed to tile the target nucleotide sequence.

4. The method of claim 1, wherein the 5′ flanking adaptor sequence or the 3′ flanking adaptor sequence is used to retrieve one or more copies of the target nucleotide sequence in the presence of the polymerase.

5. The method of claim 1, wherein the unique programmed mutation comprises one or more base changes, insertions, or deletions relative to the target nucleotide sequence.

6. The method of claim 1, wherein the unique programmed mutation is a codon swap.

7. The method of claim 1, wherein the target nucleotide sequence is a coding sequence.

8. The method of claim 1, wherein the target nucleotide sequence is more than 10 codons in length.

9. The method of claim 1, wherein each member of the set of mutagenic oligonucleotide primers is about 10 to 200 nucleotides in length.

10. A method for generating a mutant nucleotide sequence library, comprising the following steps:
a) generating, in parallel, a set of mutagenic oligonucleotide primers that are at least substantially complementary to a portion of the target nucleotide sequence, wherein each member of the set of mutagenic oligonucleotide primers comprises:
a unique programmed mutation,
a 5′ flanking adaptor sequence, and
a 3′ flanking adaptor sequence;
b) annealing and extending the set of mutagenic oligonucleotide primers to and along a wild type sense template corresponding to the target nucleotide sequence, creating a set of sense megaprimers, wherein the wild type sense template is marked for selective degradation;
c) amplifying the set of sense megaprimers using a pair of primers, wherein one of the primers recognizes and binds to the 5′ flanking adaptor sequence;
d) annealing and extending the amplified set of sense megaprimers to and along a wild type antisense template corresponding to the target nucleotide sequence, creating a mutant nucleotide sequence library, wherein:
each member of the mutant nucleotide sequence library comprises a full-length copy of the target nucleotide sequence having a unique programmed mutation derived from one member of the set of mutagenic oligonucleotide primers, and
the wild type antisense template is marked for selective degradation; and
e) amplifying the members of the mutant nucleotide sequence library.

11. The method of claim 10, wherein generating the set of mutagenic oligonucleotide primers comprises steps of
synthesizing and releasing a library of mutagenic oligonucleotides from a microarray; and
amplifying and retrieving a subset of the library of mutagenic oligonucleotides using the 3′ flanking adaptor sequence, wherein the subset is the set of mutagenic oligonucleotide primers.

12. The method of claim 10, wherein the wild type sense template and the wild type antisense template are degraded using a selective degradation agent following steps b) and d), respectively.

13. The method of claim 10, further comprising removing the 3′ flanking adaptor sequence from the set of mutagenic oligonucleotide primers after step a) and before step b).

14. The method of claim 10, further comprising removing the 5′ flanking adaptor sequence from the amplified set of sense megaprimers after step c) and before step d).

15. The method of claim 10, wherein the set of mutagenic oligonucleotide primers are designed to tile the target nucleotide sequence.

16. The method of claim 10, wherein the unique programmed mutation comprises one or more base changes, insertions, or deletions relative to the target nucleotide sequence.

17. The method of claim 10, wherein the unique programmed mutation is placed near the center of each mutagenic oligonucleotide primer.

18. The method of claim 10, wherein the target nucleotide sequence is a coding sequence.

19. The method of claim 18, wherein the coding sequence is more than 10 codons in length.

20. The method of claim 10, wherein each member of the set of mutagenic oligonucleotide primers is about 10 to 200 nucleotides in length.

21. A method for generating a mutant protein library, comprising:
generating a mutant nucleotide sequence library using the method of claim 10;
cloning each member of the mutant nucleotide sequence library into an expression plasmid; and
expressing a mutated protein from each member of the mutant nucleotide sequence library that is cloned into an expression plasmid to generate the mutant protein library.

22. The method of claim 21, wherein the mutant protein library is used to screen for the function of a mutant protein.


SEQUENCE LISTING (96 sequences)

SEQ ID NO: 1 - DNA, 16 bp, Artificial Sequence
  Synthetic  P42D (CCC>GAT)
aatgctgctggtgatg

SEQ ID NO: 2 - DNA, 16 bp, Artificial Sequence
  Synthetic  N34G (AAC>GGT), R51K (AGG>AAG)
gttctcagccgggcaa

SEQ ID NO: 3 - DNA, 16 bp, Artificial Sequence
  Synthetic  T55D (ACA>GAT)
tgttaaggagacgcga

SEQ ID NO: 4 - DNA, 16 bp, Artificial Sequence
  Synthetic  A52X (GCA>TAA)
gtaatgaaactagggt

SEQ ID NO: 5 - DNA, 16 bp, Artificial Sequence
  Synthetic  E56G (GAA>GGT)
gagtagagtcgccgga

SEQ ID NO: 6 - DNA, 16 bp, Artificial Sequence
  Synthetic  clone genotype - wildtype
tagcatacaaataaga

SEQ ID NO: 7 - DNA, 16 bp, Artificial Sequence
  Synthetic  clone genotype - wildtype
agtgtgggtggcatag

SEQ ID NO: 8 - DNA, 16 bp, Artificial Sequence
  Synthetic  in-frame deletion K18 (programmed)
acgtattaaacaacac

SEQ ID NO: 9 - DNA, 16 bp, Artificial Sequence
  Synthetic  clone genotype - wildtype
aataagtgaccggacc

SEQ ID NO: 10 - DNA, 16 bp, Artificial Sequence
  Synthetic  E8I (GAA>ATT)
ttcataaagatcacgt

SEQ ID NO: 11 - DNA, 16 bp, Artificial Sequence
  Synthetic  K33D (AAG>GAT)
tatttttaaaagtgga

SEQ ID NO: 12 - DNA, 16 bp, Artificial Sequence
  Synthetic  S47Y (TCT>TAT), E56L (GAA>TTG)
tctcagagaaatcgta

SEQ ID NO: 13 - DNA, 16 bp, Artificial Sequence
  Synthetic  I7F (ATC>TTT)
taacgttttgaatgcg

SEQ ID NO: 14 - DNA, 20 bp, Artificial Sequence
  Synthetic  clone genotype - wildtype
gcttttggtacacagcgtac

SEQ ID NO: 15 - DNA, 20 bp, Artificial Sequence
  Synthetic  E271S (GAG>TCT), A355F (GCT>TTT), A138syn (GCC>GCT)
acgtatcggaaagcaaatgc

SEQ ID NO: 16 - DNA, 20 bp, Artificial Sequence
  Synthetic  E2Q (GAG>CAG), Q167syn (CAG>CAA)
cctgagtgggcgacgcctga

SEQ ID NO: 17 - DNA, 20 bp, Artificial Sequence
  Synthetic  clone genotype - wildtype
agaagctacgtaacaaatta

SEQ ID NO: 18 - DNA, 20 bp, Artificial Sequence
  Synthetic  R202C (CGT>TGT), G245D (GGC>GAC)
tcttgcttgtgagggtgtgg

SEQ ID NO: 19 - DNA, 20 bp, Artificial Sequence
  Synthetic  K120L (AAG>TTA)
accctaagagaatacgagct

SEQ ID NO: 20 - DNA, 20 bp, Artificial Sequence
  Synthetic  S33F (TCC>TTC), E221L (GAG>TTG)
ctgcgtagaatgagcagggg

SEQ ID NO: 21 - DNA, 20 bp, Artificial Sequence
  Synthetic  F109R (TTC>CGC), K139syn (AAG>AAA)
atactcaacattctggacga

SEQ ID NO: 22 - DNA, 20 bp, Artificial Sequence
  Synthetic  L137V (CTG>GTC)
gtgcactcggggtagcaggg

SEQ ID NO: 23 - DNA, 20 bp, Artificial Sequence
  Synthetic  del 1bp frameshift (K371fs)
tggttccggactacaggaag

SEQ ID NO: 24 - DNA, 20 bp, Artificial Sequence
  Synthetic  F212L (TTT>TTA)
gccgcggggagggctagtta

SEQ ID NO: 25 - DNA, 20 bp, Artificial Sequence
  Synthetic  Q165F (CAG>TTT)
cgagacaatgcaggttagct

SEQ ID NO: 26 - DNA, 20 bp, Artificial Sequence
  Synthetic  clone genotype - wildtype
tgatatatcgcaccggagaa

SEQ ID NO: 27 - DNA, 20 bp, Artificial Sequence
  Synthetic  E271F (GAG>TTT), K373syn (AAG>AAA)
gcacatccaataccaggcgc

SEQ ID NO: 28 - DNA, 20 bp, Artificial Sequence
  Synthetic  R175H (CGC>CAC), T81syn (ACA>ACT)
ttgagtgggtcgtggcaaga

SEQ ID NO: 29 - DNA, 20 bp, Artificial Sequence
  Synthetic  G108V (GGT>GTA), G117R (GGG>AGG)
tcctgactgcaggtagaggg

SEQ ID NO: 30 - DNA, 20 bp, Artificial Sequence
  Synthetic  P36S (CCG>TCG), N247F (AAC>TTT)
gaacaatggtacctgggagc

SEQ ID NO: 31 - DNA, 20 bp, Artificial Sequence
  Synthetic  L93Y (CTG>TAC), del 1bp frameshift (S89fs)
cccaaggtgggtataaggag

SEQ ID NO: 32 - DNA, 20 bp, Artificial Sequence
  Synthetic  clone genotype - wildtype
gggaataagtaaatgggcac

SEQ ID NO: 33 - DNA, 20 bp, Artificial Sequence
  Synthetic  A84V (GCC>GTG), A88T (GCC>ACC)
gtggaaagagagggtaagaa

SEQ ID NO: 34 - DNA, 20 bp, Artificial Sequence
  Synthetic  P4I (CCG>ATT)
tggaagcgcaaagactcgag

SEQ ID NO: 35 - DNA, 20 bp, Artificial Sequence
  Synthetic  del 1bp frameshift (Q191fs), R273C (CGT>TGT), G302syn (GGG>GGA)
cgaaggtcgagtggtggaca

SEQ ID NO: 36 - DNA, 20 bp, Artificial Sequence
  Synthetic  del 1bp frameshift (I231fs), I232L (ATC>CTC)
agctaggaacgtgagaagcc

SEQ ID NO: 37 - DNA, 20 bp, Artificial Sequence
  Synthetic  syn L194CTT>CTC
ttctatgcgtgagtgaggac

SEQ ID NO: 38 - DNA, 20 bp, Artificial Sequence
  Synthetic  clone genotype - wildtype
ggtataaagggagcgggggc

SEQ ID NO: 39 - DNA, 15 bp, Artificial Sequence
  Synthetic  truncL_GAL4DBD
tacctcacgcgatct

SEQ ID NO: 40 - DNA, 15 bp, Artificial Sequence
  Synthetic  truncR_GAL4DBD
agatcaatggcaaac

SEQ ID NO: 41 - DNA, 15 bp, Artificial Sequence
  Synthetic  truncL_TP53
tgccatcttggatct

SEQ ID NO: 42 - DNA, 15 bp, Artificial Sequence
  Synthetic  truncR_TP53
agatccgagtttgtt

SEQ ID NO: 43 - DNA, 24 bp, Artificial Sequence
  Synthetic  L_TP53
gccaaagtcaacaaactcggatct

SEQ ID NO: 44 - DNA, 24 bp, Artificial Sequence
  Synthetic  R_TP53
tgtagtcagtgccatcttggatct

SEQ ID NO: 45 - DNA, 24 bp, Artificial Sequence
  Synthetic  L_GAL4DBD
cccttcacgtttgttcttggatct

SEQ ID NO: 46 - DNA, 24 bp, Artificial Sequence
  Synthetic  R_GAL4DBD
aggctatgggacttaaagggatct

SEQ ID NO: 47 - DNA, 23 bp, Artificial Sequence
  Synthetic  R_TP53_U
tgtagtcagtgccatcttggatc

SEQ ID NO: 48 - DNA, 23 bp, Artificial Sequence
  Synthetic  R_GAL4DBD_U
aggctatgggacttaaagggatc

SEQ ID NO: 49 - DNA, 23 bp, Artificial Sequence
  Synthetic  L_TP53_U
gccaaagtcaacaaactcggatc

SEQ ID NO: 50 - DNA, 23 bp, Artificial Sequence
  Synthetic  L_GAL4DBD_U
cccttcacgtttgttcttggatc

SEQ ID NO: 51 - DNA, 40 bp, Artificial Sequence
  Synthetic  GAL4_CLONE_F
actagtggatcccccgacagagaagcaagcctcctgaaag

SEQ ID NO: 52 - DNA, 48 bp, Artificial Sequence
  Synthetic  GAL4_CLONE_R
aggtcgacggtatcggcggccgcggggtttttcagtatctacgattca

SEQ ID NO: 53 - DNA, 68 bp, Artificial Sequence
  Synthetic  GAL4_NTERM_R
ataactaattacatgactcgaggtcgacggtatcgtcatctattcagaacccattattgttggggtcc

SEQ ID NO: 54 - DNA, 42 bp, Artificial Sequence
  Synthetic  GAL4_SENSE_F
cgttacagttctgcgattgatccaagcgcgcaattaaccctc

SEQ ID NO: 55 - DNA, 20 bp, Artificial Sequence
  Synthetic  GAL4_SENSE_R
aaatccaacggaattgtgga

SEQ ID NO: 56 - DNA, 59 bp, Artificial Sequence
  Synthetic  GAL4_ANTISENSE_F
acgatctatccagattcatgcactactacagcatcagtacgacacatgatcatatggca

SEQ ID NO: 57 - DNA, 22 bp, Artificial Sequence
  Synthetic  GAL4_ANTISENSE_R
gaacccattattgttggggtcc

SEQ ID NO: 58 - DNA, 22 bp, Artificial Sequence
  Synthetic  OUTER_F
cgttacagttctgcgattgatc

SEQ ID NO: 59 - DNA, 48 bp, Artificial Sequence
  Synthetic  GAL4_OUTER_R
aggtcgacggtatcgtcatctattcagaacccattattgttggggtcc

SEQ ID NO: 60 - DNA, 20 bp, Artificial Sequence
  Synthetic  P53_SENSE_F
caagtctccaccccattgac

SEQ ID NO: 61 - DNA, 58 bp, Artificial Sequence
  Synthetic  P53_SENSE_R
acgatctatccagattcatgcactactacagcatcagtctctcgtcgctctccatctc

SEQ ID NO: 62 - DNA, 60 bp, Artificial Sequence
  Synthetic  P53_ANTISENSE_F
gtcagcctctaatggctcgtatgatagtgcagccgctggtcaccaaaatcaacgggactt

SEQ ID NO: 63 - DNA, 20 bp, Artificial Sequence
  Synthetic  P53_ANTISENSE_R
cctcgtagcggtagctgaag

SEQ ID NO: 64 - DNA, 43 bp, Artificial Sequence
  Synthetic  SA_REV_BCFWD
actttatcaatctcgctccaaaccagctccacgaggcaaatgg

SEQ ID NO: 65 - DNA, 46 bp, Artificial Sequence
  Synthetic  SA_REV_BCREV
actttatcaatctcgctccaaacctatggtcaatcgtgcatcacgc

SEQ ID NO: 66 - DNA, 45 bp, Artificial Sequence
  Synthetic  GAL4_SA_1F
ctaaatggctgtgagagagctcagggtcttctcgaggaaaaatca

SEQ ID NO: 67 - DNA, 22 bp, Artificial Sequence
  Synthetic  GAL4_SA_2F
gaacccattattgttggggtcc

SEQ ID NO: 68 - DNA, 25 bp, Artificial Sequence
  Synthetic  GAL4_SA_3F
gacagagaagcaagcctcctgaaag

SEQ ID NO: 69 - DNA, 19 bp, Artificial Sequence
  Synthetic  P53_SA_1F
tgatgcggcactcgatctc

SEQ ID NO: 70 - DNA, 20 bp, Artificial Sequence
  Synthetic  P53_SA_2F
aattcgtcgactggatccgg

SEQ ID NO: 71 - DNA, 62 bp, Artificial Sequence
  Synthetic  NEXV2_AD1
aatgatacggcgaccaccgagatctacactcgtcggcagcgtcagatgtgtataagagacag

SEQ ID NO: 72 - DNA, 67 bp, Artificial Sequence
  Synthetic  Index 1
caagcagaagacggcatacgagattacgaagtcgaccgtcggcactttatcaatctcgctccaaacc

SEQ ID NO: 73 - DNA, 67 bp, Artificial Sequence
  Synthetic  Index 2
caagcagaagacggcatacgagatgacgagattgaccgtcggcactttatcaatctcgctccaaacc

SEQ ID NO: 74 - DNA, 67 bp, Artificial Sequence
  Synthetic  Index 3
caagcagaagacggcatacgagataccgtaagagaccgtcggcactttatcaatctcgctccaaacc

SEQ ID NO: 75 - DNA, 67 bp, Artificial Sequence
  Synthetic  Index 4
caagcagaagacggcatacgagattagtggcaagaccgtcggcactttatcaatctcgctccaaacc

SEQ ID NO: 76 - DNA, 67 bp, Artificial Sequence
  Synthetic  Index 5
caagcagaagacggcatacgagatcattaacgcgaccgtcggcactttatcaatctcgctccaaacc

SEQ ID NO: 77 - DNA, 67 bp, Artificial Sequence
  Synthetic  Index 6
caagcagaagacggcatacgagattcgttgaaggaccgtcggcactttatcaatctcgctccaaacc

SEQ ID NO: 78 - DNA, 67 bp, Artificial Sequence
  Synthetic  Index 7
caagcagaagacggcatacgagataagcgttcagaccgtcggcactttatcaatctcgctccaaacc

SEQ ID NO: 79 - DNA, 67 bp, Artificial Sequence
  Synthetic  Index 8
caagcagaagacggcatacgagatcgcaagcgtgaccgtcggcactttatcaatctcgctccaaacc

SEQ ID NO: 80 - DNA, 67 bp, Artificial Sequence
  Synthetic  Index 9
caagcagaagacggcatacgagatgcagcgcgagaccgtcggcactttatcaatctcgctccaaacc

SEQ ID NO: 81 - DNA, 67 bp, Artificial Sequence
  Synthetic  Index 10
caagcagaagacggcatacgagatcgcgcagctgaccgtcggcactttatcaatctcgctccaaacc

SEQ ID NO: 82 - DNA, 67 bp, Artificial Sequence
  Synthetic  Index 11
caagcagaagacggcatacgagattcaagcgcagaccgtcggcactttatcaatctcgctccaaacc

SEQ ID NO: 83 - DNA, 67 bp, Artificial Sequence
  Synthetic  Index 12
caagcagaagacggcatacgagatcagtcgcaggaccgtcggcactttatcaatctcgctccaaacc

SEQ ID NO: 84 - DNA, 67 bp, Artificial Sequence
  Synthetic  Index 13
caagcagaagacggcatacgagatgcgtcagttgaccgtcggcactttatcaatctcgctccaaacc

SEQ ID NO: 85 - DNA, 67 bp, Artificial Sequence
  Synthetic  Index 14
caagcagaagacggcatacgagatagtcgcgcagaccgtcggcactttatcaatctcgctccaaacc

SEQ ID NO: 86 - DNA, 43 bp, Artificial Sequence
  Synthetic  GAL4_BC_AMP_F
ctaaatggctgtgagagagctcagagctccacgaggcaaatgg

SEQ ID NO: 87 - DNA, 46 bp, Artificial Sequence; identical to SEQ ID NO: 65
  Synthetic  GAL4_BC_AMP_R

SEQ ID NO: 88 - DNA, 61 bp, Artificial Sequence
  Synthetic  ILMN_P5_SA
aatgatacggcgaccaccgagatctacacacgtaggcctaaatggctgtgagagagctcag

SEQ ID NO: 89 - DNA, 68 bp, Artificial Sequence
  Synthetic  P416CYC_BC_CAS
ctcgagtctagaagctccacgaggcaaatggnnnnnnnnnnnnnnnngcgtgatgcacgattgaccat

SEQ ID NO: 90 - DNA, 38 bp, Artificial Sequence
  Synthetic  P416CYC_AGEMFE_TOP
caccggtgcatgtctggctttaaaattcaattgggtac

SEQ ID NO: 91 - DNA, 38 bp, Artificial Sequence
  Synthetic  P416CYC_AGEMFE_BTM
ccaattgaattttaaagccagacatgcaccggtggtac

SEQ ID NO: 92 - DNA, 39 bp, Artificial Sequence
  Synthetic  P416CYC_AMP_BC_CAS_F
ggccggtaccaccggtctcgagtctagaagctccacgag

SEQ ID NO: 93 - DNA, 36 bp, Artificial Sequence
  Synthetic  P416CYC_AMP_BC_CAS_R
aattgggtaccaattggagctcggatcctatggtca

SEQ ID NO: 94 - DNA, 61 bp, Artificial Sequence
  Synthetic  P53_BC_CAS
agctccacgaggcaaatggnnnnnnnnnnnnnnnnnnnngcgtgatgcacgattgaccata

SEQ ID NO: 95 - DNA, 66 bp, Artificial Sequence
  Synthetic  P53_AMP_BC_CAS_F
ggacgtccagacacagcataggctacctggccatgcccagcggccgcagctccacgaggcaaatgg

SEQ ID NO: 96 - DNA, 68 bp, Artificial Sequence
  Synthetic  P53_AMP_BC_CAS_R
gcatgagaggacagtgccaagcaagcaactcaaatgtcccgaattctatggtcaatcgtgcatcacgc